(19) United States
(12) Patent Application Publication
(10) Pub. No.: US 2026/0015601 A1
(43) Pub. Date: Jan. 15, 2026

(54) IMPROVED LOADING OF MOLECULES AND COMPLEXES TO REACTION SITES

(71) Applicant: Pacific Biosciences of California, Inc., Menlo Park, CA (US)

(72) Inventors: Carl WASSERMANN, Menlo Park, CA (US); Richa Pathak, Menlo Park, CA (US); Aparna Vedula, Menlo Park, CA (US); Alexander Plumridge, Menlo Park, CA (US); Thang Pham, Menlo Park, CA (US)

(21) Appl. No.: 19/268,430

(22) Filed: Jul. 14, 2025

Related U.S. Application Data
(60) Provisional application No. 63/671,669, filed on Jul. 15, 2024.

(51) Int. Cl. C12N 11/08; C12N 9/12

(52) U.S. Cl. CPC C12N 11/08; C12N 9/1241

(57) ABSTRACT

Disclosed are methods for directly loading samples to reaction sites on a surface of a substrate without any step of prewetting the surface. The methods result in a reduced sample input amount by at least 2- to 10-fold compared to the conventional protocol that requires the prewetting step.

### CROSS REFERENCE TO RELATED APPLICATIONS

This application claims the benefit of U.S. Provisional Appl. No. 63/671,669, filed Jul. 15, 2024, the full disclosure of which is hereby incorporated by reference herein in its entirety for all purposes.

### BACKGROUND OF THE INVENTION

Techniques in molecular biology and molecular medicine often rely on analysis of single biological molecules. Such techniques include DNA and RNA sequencing, polymorphism detection, detection of proteins of interest, detection of protein-nucleic acid complexes, and many others. The high sensitivity, high throughput, and low reagent costs of single molecule analysis make it an increasingly attractive approach for a variety of detection and analysis problems in molecular medicine, from low-cost genomics to high sensitivity marker analysis.

The small observation volumes often used for single molecule analysis methods are typically provided by immobilizing or otherwise localizing molecules of interest within an optical confinement reaction/observation region, such as in an array of extremely small wells (e.g., an array of zero mode waveguides (ZMWs)), and delivering molecules of interest (including, for e.g., one or more templates, primers, enzymes, etc.) to the reaction region. It is desirable to develop methods and compositions to further reduce sample input, e.g., sample amount and sample volume, while maintaining or improving the performance of single molecule analyses. The present disclosure addresses these needs.

### SUMMARY OF THE INVENTION

The present disclosure provides methods, devices, compositions, and systems for distribution of molecules of interest into reaction sites. In particular, the methods, devices, compositions, and systems disclosed herein reduce the sample amount or sample volume required for loading molecules of interest into reaction sites by 2-fold to 10-fold without compromising the performance of sequencing analyses.

In one aspect, the present disclosure provides a method of loading polymerase enzyme complexes into a plurality of nanoscale wells, the method comprising contacting a loading solution to a surface of a substrate comprising a plurality of nanoscale wells, wherein the loading solution comprises (a) one or more polymerase enzyme complexes comprising a template nucleic acid and a polymerase enzyme; (b) one or more nonionic surfactants; and (c) one or more crowding agents.

In some embodiments, the surface is hydrophobic before contacting with the loading solution. In some embodiments, the surface is not prewetted with any buffer or surfactant before contacting with the loading solution. In some embodiments, the surface is functionalized before contacting with the loading solution.

In a related aspect, this disclosure provides a loading solution for loading polymerase enzyme complexes into a plurality of nanoscale wells, the loading solution comprising (a) one or more nonionic surfactants and (b) one or more crowding agents. In some embodiments, the loading solution further comprises one or more polymerase enzyme complexes comprising a template nucleic acid and a polymerase enzyme.

In some embodiments, the nonionic surfactant in the loading solution is selected from the group consisting of Triton X-100, EcoSurf, Tergitol, poloxamer, ECO Brij, Brij, n-Dodecyl 3-D-maltoside, N,N-dimethyldodecylamine N-oxide, and Tween-20. In some embodiments, the loading solution comprises about 0.01% (v/v), about 0.02% (v/v), about 0.03% (v/v), about 0.04% (v/v), about 0.05% (v/v), about 0.06% (v/v), about 0.07% (v/v), about 0.08% (v/v), about 0.09% (v/v), about 0.1% (v/v), about 0.15% (v/v), about 0.2% (v/v), about 0.25% (v/v), about 0.3% (v/v), about 0.35% (v/v), about 0.4% (v/v), about 0.45% (v/v), about 0.5% (v/v), about 0.55% (v/v), about 0.6% (v/v), about 0.65% (v/v), about 0.7% (v/v), about 0.75% (v/v), about 0.8% (v/v), about 0.85% (v/v), about 0.9% (v/v), about 0.95% (v/v), or about 1% (v/v) of the one or more nonionic surfactants.

In some embodiments, the polymerase enzyme complex comprises a template nucleic acid complexed with a polymerase enzyme. In some embodiments, the complex further comprises a primer hybridized to the template nucleic acid. In some embodiments, the one or more polymerase enzyme complexes are suspended in a sample mix solution, which comprises one or more surfactants. In some embodiments, the sample mix solution comprises one or more nonionic surfactants such as a surfactant selected from the group consisting of Triton X-100, EcoSurf, Tergitol, poloxamer, ECO Brij, Brij, n-Dodecyl β-D-maltoside, N,N-dimethyldodecylamine N-oxide, and Tween-20. In some embodiments, the nonionic surfactant is a detergent. In some embodiments, the sample mix solution further comprises one or more viscosity adjusting agents such as glycerol, a low molecular weight PEG, a polysaccharide such as cellulose, agar, dextrin, or trehalose, and polyvinylpyrrolidone (PVP). In some embodiments, the sample mix solution further comprises one or more monovalent cations such as Na⁺ and K⁺, one or more divalent cations such as Sr²⁺, or a combination thereof. In some embodiments, the monovalent cation is at a concentration of 50 to 500 mM or 100 to 300 mM in the sample mix solution. In some embodiments, the divalent cation is at a concentration of 0.05 to 10 mM in the sample mix solution.

In some embodiments, the one or more crowding agents comprise a high molecular weight PEG, polyvinylpyrrolidone (PVP), or a combination thereof. In some embodiments, the high molecular weight PEG has a molecular weight between about 3,000 g/mol and about 40,000 g/mol, between about 5,000 g/mol and about 30,000 g/mol, between about 6,000 g/mol and about 20,000 g/mol, between about 7,000 g/mol and about 12,000 g/mol, or between about 8,000 g/mol and about 10,000 g/mol. In some embodiments, the high molecular weight PEG is selected from the group consisting of PEG 3000, PEG 4000, PEG 5000, PEG 6000, PEG 7000, PEG 8000, PEG 9000, PEG 10000, and PEG 20000. In some embodiments, the PEG is present in the loading solution at a concentration of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM.

In some embodiments, the loading solution further comprises one or more viscosity adjusting agents at a concentration of about 0.1% (v/v), about 0.5% (v/v), about 1% (v/v), about 1.5% (v/v), about 2% (v/v), about 2.5% (v/v), about 3% (v/v), about 3.5% (v/v), about 4% (v/v), about 4.5% (v/v), about 5% (v/v), about 5.5% (v/v), about 6% (v/v), about 6.5% (v/v), about 7% (v/v), about 7.5% (v/v), about 8% (v/v), about 8.5% (v/v), about 9% (v/v), about 9.5% (v/v), or about 10% (v/v). In some embodiments, the viscosity adjusting agent is selected from the group consisting of glycerol, a low molecular weight PEG, a polysaccharide such as cellulose, agar, dextrin, or trehalose, and polyvinylpyrrolidone (PVP). In some embodiments, the low molecular weight PEG has a molecular weight between 200 g/mol and 1000 g/mol, such as about 200 g/mol, about 300 g/mol, about 400 g/mol, about 500 g/mol, about 600 g/mol, about 700 g/mol, about 800 g/mol, about 900 g/mol, and about 1000 g/mol.

In some embodiments, the loading solution further comprises one or more monovalent cations, one or more divalent cations, or a combination thereof. In some embodiments, the monovalent cations include Na⁺ such as sodium acetate (NaOAc) and/or K⁺ such as potassium acetate (KOAc). In some embodiments, the divalent cation is Sr²⁺ such as strontium acetate (SrOAc). In some embodiments, K⁺ such as KOAc is at a concentration of about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM in the loading solution. In some embodiments, Sr²⁺ such as SrOAc is at a concentration of about 10 μM, about 20 μM, about 30 μM, about 40 μM, about 50 μM, about 60 μM, about 70 μM, about 80 μM, about 90 μM, about 100 μM, about 110 μM, about 120 μM, about 130 μM, about 140 μM, about 150 μM, about 160 μM, about 170 μM, about 180 μM, about 190 μM, about 200 μM, about 210 μM, about 220 μM, about 230 μM, about 240 μM, about 250 μM, about 260 μM, about 270 μM, about 280 μM, about 290 μM, or about 300 μM in the loading solution.

In yet another related aspect, this disclosure provides a method of sequencing a nucleic acid, comprising the steps of loading one or more nucleic acid and polymerase enzyme complex into array regions as disclosed herein and analyzing the nucleic acid in the array regions by determining its sequence. In some embodiments, the sequencing method is single-molecule nucleic acid sequencing.

### BRIEF DESCRIPTION OF THE DRAWINGS

This application contains at least one drawing executed in color. Copies of this application with color drawing(s) will be provided by the Office upon request and payment of the necessary fees.

FIG. 1: The loading heat maps and the base rate density graphs show comparison of sample direct loading using different surfactants.

FIG. 2: The loading heatmaps and base rate density graphs show that various concentrations of Tergitol ranging from 0.25% (v/v) to 0.45% (v/v) in a loading solution comprising PEG 8000 achieved good loading results.

FIG. 3: The HiFi insert size distribution shows that various concentrations of Tergitol ranging from 0.25% (v/v) to 0.45% (v/v) in a loading solution comprising PEG 8000 achieved good loading results.

FIGS. 4A and 4B: The loading heat maps show that various concentrations of Tergitol ranging from 0.01% (v/v) to 0.5% (v/v) resulted in a loading ranging from 6.1% to 42.0% (4A), and that various concentrations of EcoSurf ranging from 0.1% (v/v) to 0.5% (v/v) resulted in a loading ranging from 29.8% to 37.6% (4B).

FIG. 5A shows loading heatmaps for all tested conditions. FIG. 5B presents aligned polymerase read length distributions and polymerase survival curves. FIG. 5C compares replicate results for Tergitol 15-S-7 and Tergitol 15-S-9, highlighting variability. FIG. 5D illustrates photonic recovery via SNR vs. PkMid analysis for all conditions.

### DETAILED DESCRIPTION OF INVENTION

The practice of the present disclosure may employ, unless otherwise indicated, conventional techniques and descriptions of organic chemistry, polymer technology, molecular biology (including recombinant techniques), cell biology, biochemistry, and immunology, which are within the skill of the art. Such conventional techniques include nucleic acid synthesis, isolation and/or manipulation, polymer array synthesis, hybridization, ligation, phage display, and detection using a label. Specific illustrations of suitable techniques can be had by reference to the examples herein below. However, other equivalent conventional procedures can, of course, also be used. Such conventional techniques and descriptions can be found in standard laboratory manuals such as Sambrook et al., Molecular Cloning—A Laboratory Manual (3rd Ed.), Vol. 1-3, Cold Spring Harbor Laboratory, Cold Spring Harbor, N.Y., 2000, Current Protocols in Molecular Biology, F. M. Ausubel et al., eds., Current Protocols, a joint venture between Greene Publishing Associates, Inc. and John Wiley & Sons, Inc., (supplemented through 2018), Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, TRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

Unless defined otherwise, all technical and scientific terms used herein have the same meaning as commonly understood by one of ordinary skill in the art to which this disclosure belongs. All publications mentioned herein are incorporated herein by reference for the purpose of describing and disclosing devices, compositions, formulations and methodologies which are disclosed in the publication and which might be used in connection with the presently disclosed technology.

Where a range of values is provided, it is understood that each intervening value, to the tenth of the unit of the lower limit unless the context clearly dictates otherwise, between the upper and lower limit of that range and any other stated or intervening value in that stated range is encompassed within this disclosure. The upper and lower limits of these smaller ranges may independently be included in the smaller ranges also encompassed within this disclosure, subject to any specifically excluded limit in the stated range. Where the stated range includes one or both of the limits, ranges excluding either or both of those included limits are also included in this disclosure.

In the following description, numerous specific details are set forth to provide a more thorough understanding of this disclosure. However, it will be apparent to one of skill in the art that this disclosure may be practiced without one or more of these specific details. In other instances, features and procedures well known to those skilled in the art have not been described to avoid obscuring the invention.

All numerical designations, e.g., pH, temperature, time, concentration, and molecular weight, including ranges, are approximations which are varied (+) or (−) by increments of 0.1. It is to be understood, although not always explicitly stated, that all numerical designations are preceded by the term “about.” The term “about” as used herein indicates the value of a given quantity varies by +/−10% of the value, or optionally +/−5% of the value, or in some embodiments, by +/−1% of the value so disclosed. The term “about” also includes the exact value “X” in addition to minor increments of “X” such as “X+0.1” or “X−0.1.” It also is to be understood, although not always explicitly stated, that the reagents disclosed herein are merely exemplary and that equivalents of such are known in the art.

“Nucleic acid,” “polynucleotide,” “oligonucleotide,” or grammatical equivalents herein means at least two nucleotides covalently linked together. A nucleic acid of this disclosure will generally contain phosphodiester bonds, although in some cases, nucleic acid analogs are included that may have alternate backbones, comprising, for example, phosphoramide, phosphorothioate, phosphorodithioate, and peptide nucleic acid backbones and linkages. Other analog nucleic acids include those with positive backbones, non-ionic backbones, and non-ribose backbones, including those disclosed in U.S. Pat. Nos. 5,235,033 and 5,034,506 (“Uncharged morpholino-based polymers having achiral intersubunit linkages”). The nucleic acid may have other modifications, such as the inclusion of heteroatoms, the attachment of labels, such as dyes, or substitution with functional groups which will still allow for base pairing and for recognition by an enzyme. The length of a nucleic acid can be indicated in either nucleotides (measured on one strand of a single or double stranded nucleic acid) or base pairs (measured on both strands of a nucleic acid that is or that can be double stranded if hybridized to a complementary strand); units of nucleotides and base pairs thus can be used interchangeably to refer to an identical length, as will be clear to one skilled in the art.

### I. Overview

The present disclosure is directed to methods, devices, compositions and systems for distributing enzyme molecules (and any molecules or compounds associated with those enzyme molecules) into a plurality of array regions. In general, the methods, devices, compositions and systems disclosed herein result in improved loading of compositions to a surface as compared to typical loading methods that require prewetting the surface. Note that although for ease of discussion this disclosure generally refers to polymerase enzymes and polymerase compositions, it will be appreciated that any other molecules, including for example other enzymes, other proteins, or nucleic acids, can be used in the methods, devices, compositions, and systems disclosed herein. In other words, any of the loading methods disclosed herein can be used to load nucleic acids alone, enzymes alone, or any combination of enzymes and nucleic acids, including polymerase enzymes complexed with a nucleic acid template. “Polymerase compositions” as used herein is meant to encompass compositions comprising polymerase enzymes as well as any associated molecules, including for example nucleic acid templates and primer sequences. In certain examples, the polymerase compositions comprise polymerase complexes in which a polymerase is attached to a nucleic acid template that is in some examples also further hybridized to a primer. The polymerase-template complexes can be immobilized in the array regions, for example, at the bottom of nanoscale wells, e.g., by binding to a moiety located at the bottom of each well.

Techniques for single molecule analysis typically are applied to analyze a very small sample amount. While this is overall a benefit of these techniques, it is challenging to load samples of very limited amount and/or very limited volume to an array of reaction sites. The technology disclosed herein achieves optimal loading with reduced sample input.

Typically, single molecule analyses are carried out in a microfluidic device such as a flow cell, which enables controlled delivery of reagents over a functional surface of a substrate, where the reactions or detections take place. The substrate includes one or more reaction sites or regions, which may be engineered into suitable structural and functional forms, including but not limited to nanowells, nanopores and nanoFETs. Conventionally, it is required to pre-wet the hydrophobic surface of the flow cell with one or more surfactants before sample delivery. Omitting this prewetting step impacts loading, loading uniformity, sequencing rate, and signal to noise ratio (SNR). However, prewetting requires additional washes to remove the surfactants and a greater sample volume is required to sufficiently load the flow cell. The present disclosure provides methods, compositions, and systems for distribution of molecules of interest into a plurality of reaction sites on a flow cell. The disclosed technology eliminates the steps of prewetting the surface and washing the surface after prewetting, while allowing the use of a reduced amount of sample input for loading.

For any of the loading methods disclosed in the sections below, the reaction sites will in some aspects comprise an array of reaction sites, including an array of nanoscale wells, and the molecules of interest include polymerase enzyme complexes, where the complexes comprise template nucleic acids complexed with polymerase enzymes. For ease of discussion, the majority of the disclosure herein is directed to the loading of an array of nanoscale wells (also referred to herein as “nanowells”) with template nucleic acids and/or complexes that include template nucleic acids, but it will be appreciated that any of the methods disclosed herein are applicable to other types of reaction sites and other types of molecules. In some examples, such nanowells may include but are not limited to zero mode waveguides (ZMWs). In further examples, those ZMWs may have biotionylated bases and passivated sides, which can be of use in the methods of loading disclosed herein as well as in later downstream applications, such as sequencing reactions. As will be appreciated, any discussion herein referring to nanoscale wells and/or ZMWs is applicable to any form of reaction sites and encompass all types of surfaces, shapes and configurations of regions into which molecules of interest can be loaded.

In general, the methods disclosed herein include delivering template nucleic acids to the array in a loading solution. In some examples, the loading solution includes one or more of: template nucleic acids, polymerase enzymes, primers, and nucleotides. In some examples, the template nucleic acids, polymerase enzymes, and primers are present in die loading solution as a complex that includes a primer hybridized to the template nucleic acid, and the nucleotides are present in the nanoscale wells either through the loading solution or separately from the loading solution. The nucleotides can be labeled or otherwise capable of generating a signal

For any of the loading methods disclosed herein, the array of nanoscale wells can be part of a substrate that is configured to allow detection of signals only from molecules within the wells themselves. In such a configuration, even if signals are being generated throughout the loading process, those signals will not be detected unless a complex is located within the nanoscale well itself. Such substrates include substrates of ZMWs, such as those disclosed in U.S. Pat. Nos. 9,624,540, 9,372,308, 9,223,084, 8,994,946, 8,906,670, 8,993,307, 8,802,600, 8,471,230, 7,907,800, 7,820,983, 7,302,146, and 6,917,726, the contents of which are incorporated herein by reference in their entirety for all purposes and in particular for all teachings related to substrates and arrays of nanoscale wells. In some examples, the loading methods disclosed herein deliver molecules of interest, such as polymerase enzyme complexes, to the observation volume of the nanoscale wells. The “observation volume” generally refers to that volume of the nanoscale wells that is observable by whatever detection methods used to detect signals from the wells. For example, in the case of fluorescence-based detection, it is that volume which is exposed to excitation radiation and/or from which emission radiation is gathered by an adjacent optical train/detector. In some embodiments, the observation volume is an extremely small volume proximal to the base of a nanoscale well, e.g., a ZMW. See, e.g., U.S. Pat. Nos. 7,906,284 and 6,917,726, hereby incorporated by reference in their entireties, in particular for all teachings related to Z MWs

Once the polymerase enzyme complexes are loaded, detection of the complex can then be accomplished by monitoring signals from the labeled analog. For example, during sequencing by incorporation, e.g., single molecule sequencing by synthesis (SMS), nucleotide (or nucleotide analog) incorporation events are detected in real-time as the bases are incorporated into the extension product. This can be accomplished by immobilizing a synthesis complex, which includes a polymerase enzyme, such as a DNA polymerase enzyme, a template nucleic acid sequence, and a primer sequence that is complementary to a portion of the template sequence, within an optically confined space (e.g., an observation volume) or otherwise resolved as an individual molecular complex. Some SMS methods employ nucleotide analogs that include fluorescent labels coupled to the polyphosphate chain of the analog, which are then exposed to the complex. Upon incorporation, the nucleotide-along with its fluorescent label—is retained by the complex for a time and in a manner that permits the detection by a sequencing system of a signal “pulse” from the fluorescent label at the incorporation site. The sequentially detected signal pulses are then interpreted by the sequencing system to generate a readout corresponding to the sequence of the template nucleic acid. For a discussion of preferred sequence by incorporation processes, see, e.g., U.S. Pat. Nos. 6,056,661, 7,052,847, 7,033,764, 7,056,676, 7,361,466, 8,133,672, and 8,182,993, the full disclosures of which are hereby incorporated herein by reference in their entirety for all purposes. Detection of signal pulses during loading can be achieved as disclosed for sequencing by incorporation, although it will be evident that pulses need not be interpreted to generate nucleotide sequence information where only level of loading is desired to be monitored.

In some embodiments, the nucleotide analog and a component of the polymerase complex (e.g., the polymerase) bear labeling components that interact via FRET to produce a signal only when the labeling components are in close proximity (e.g., during incorporation of the analog). In other embodiments, the nucleotide analog bears a label that is capable of generating a signal regardless of the label's proximity to the complex (e.g., a fluorescent label). Although a fluorescently labeled nucleotide analog can generate a signal whenever exposed to excitation light, in preferred embodiments, detectable signals are generated only within the observation volume of a nanoscale well.

II. Loading with Low Sample Input

In certain aspects, this disclosure is directed to a method for directly loading molecules of interest, including but not limited to polymerase enzyme complexes, and nucleic acid molecules, into a plurality of array regions such as nanoscale wells. The method does not require prewetting the surface comprising a plurality of nanoscale wells with one or more surfactants. Compared to the conventional loading protocol that requires a prewetting step, the disclosed method results in at least 2- to 10-fold reduction in the sample amount. The method comprises contacting a loading solution to the surface comprising a plurality of nanoscale wells, wherein the surface is not prewetted, and wherein the loading solution comprises: (a) one or more polymerase enzyme complexes comprising a template nucleic acid and a polymerase enzyme, (b) one or more surfactants such as nonionic surfactants, and (c) one or more crowding agents such as a PEG having a high molecular weight and polyvinylpyrrolidone (PVP). Upon contact, the loading solution is allowed to diffuse freely over the surface of the substrate such that the one or more polymerase enzyme complexes are distributed into the plurality of nanoscale wells. Alternatively, the movement of the loading solution over the surface can be assisted, e.g., by pressure, pulling/drawing forces, pushing forces, density gradient, and/or concentration gradient. Various loading methods are disclosed in U.S. Pat. No. 10,814,299, incorporated herein by reference in its entirety for all purposes. In some embodiments, the array regions comprise nanoscale wells comprising a coupling agent at their bases, and the polymerase-template complexes diffuse through the solution to the bases of the nanoscale wells and bind to the coupling agent, thereby immobilizing the polymerase-template complexes in the nanoscale wells.

In some embodiments, the loading solution further comprises one or more viscosity adjusting agent. In some embodiments, the one or more polymerase enzyme complexes are suspended in a sample mix solution before mixing with the loading solution. In some embodiments, the sample mix solution comprises one or more surfactants such as nonionic surfactants. In some embodiments, the sample mix solution comprises one or more viscosity adjusting agents.

In some embodiments, the surfactant has an HLB value ranging between 10 and 18. In some embodiments, the surfactant has an HLB value of about 10, about 11, about 12, about 13, about 14, about 15, about 16, about 17, or about 18. In some embodiments, the nonionic surfactant is a detergent. In some embodiments, the loading solution comprises about 0.01% (v/v), about 0.02% (v/v), about 0.03% (v/v), about 0.04% (v/v), about 0.05% (v/v), about 0.06% (v/v), about 0.07% (v/v), about 0.08% (v/v), about 0.09% (v/v), about 0.1% (v/v), about 0.15% (v/v), about 0.2% (v/v), about 0.25% (v/v), about 0.30% (v/v), about 0.35% (v/v), about 0.4% (v/v), about 0.45% (v/v), about 0.5% (v/v), about 0.55% (v/v), about 0.6% (v/v), about 0.65% (v/v), about 0.7% (v/v), about 0.75% (v/v), about 0.8% (v/v), about 0.85% (v/v), about 0.9% (v/v), about 0.95% (v/v), or about 1% (v/v) of the nonionic surfactant. In some embodiments, the nonionic surfactant is selected from the group consisting of Triton X-100 (polyethylene glycol tert-octylphenyl ether, CAS 9002-93-1), EcoSurf (including but not limited to EcoSurf EH-9 (2-Ethylhexanol ethoxylate (average 9 EO units), CAS 64366-70-7)), Tergitol (including but not limited to Tergitol-15-S-7 (secondary alcohol ethoxylate (7 EO units), CAS 84133-50-6), Tergitol-15-S-8 (secondary alcohol ethoxylate (8 EO units), CAS 84133-50-6), Tergitol-15-S-9 (secondary alcohol ethoxylate (9 EO units), CAS 68131-40-8), Tergitol-15-S-12 (secondary alcohol ethoxylate (12 EO units), CAS 84133-50-6), Tergitol-15-5-15 (secondary alcohol ethoxylate (15 EO units), CAS 84133-50-6), Tergitol-15-S-20 (secondary alcohol ethoxylate (20 EO units), CAS 84133-50-6), Tergitol-15-S-30 (secondary alcohol ethoxylate (30 EO units), CAS 84133-50-6), Tergitol-15-S-40 (secondary alcohol ethoxylate (40 EO units), CAS 84133-50-6), Tergitol TMN6 (polyethylene glycol trimethylnonyl ether (6 EO units), CAS 60828-78-6), Tergitol TMN9 (polyethylene glycol trimethylnonyl ether (9 EO units), CAS 60828-78-6), Tergitol TMN10 (polyethylene glycol trimethylnonyl ether (10 EO units), CAS 60828-78-6), and Tergitol TMN100X (polyethylene glycol trimethylnonyl ether (100 EO units), CAS 60828-78-6)), poloxamers (including but not limited to Pluronic F68 ((poly(ethylene glycol)-block-poly(propylene glycol)-block-poly(ethylene glycol), CAS 9003-11-6)), Pluronic F88 (poly(ethylene glycol)-block-poly(propylene glycol)-block-poly(ethylene glycol), CAS 9003-11-6), Pluronic F98 (poly(ethylene glycol)-block-poly(propylene glycol)-block-poly(ethylene glycol), CAS 9003-11-6), Pluronic F108 (poly(ethylene glycol)-block-poly(propylene glycol)-block-poly(ethylene glycol), CAS 9003-11-6), Pluronic F127 (poly(ethylene glycol)-block-poly(propylene glycol)-block-poly(ethylene glycol), CAS 9003-11-6), and Pluronic F137 (poly(ethylene glycol)-block-poly(propylene glycol)-block-poly(ethylene glycol), CAS 9003-11-6)), ECO Brij products (including but not limited to ECO Brij 02 (polyoxyethylene (2) oleyl ether, CAS 9004-98-2), ECO Brij 03 (polyoxyethylene (3) oleyl ether, CAS 9004-98-2), ECO Brij 05 (polyoxyethylene (5) oleyl ether, CAS 9004-98-2), ECO Brij 010 (polyoxyethylene (10) oleyl ether, CAS 9004-98-2), and ECO Brij 020 (polyoxyethylene (20) oleyl ether, CAS 9004-98-2)), Brij products (including but not limited to Brij 35 (polyoxyethylene (23) lauryl ether, CAS 9002-92-0), Brij 56 (polyoxyethylene (10) cetyl ether, CAS 9004-95-9), Brij 58 (Polyoxyethylene (20) cetyl ether, CAS 9004-95-9), and Brij 93 (polyoxyethylene (2) oleyl ether, CAS 9004-98-2)), n-Dodecyl β-D-maltoside (CAS 69227-93-6), N,N-dimethyldodecylamine N-oxide (CAS 1643-20-5), and Tween-20 (polyoxyethylene (20) sorbitan monolaurate, CAS 9005-64-5).

In some embodiments, the crowding agent is a high molecular weight PEG. A variety of PEGs are known in the art and are suitable for use in the methods. In some embodiments, the PEG has a molecular weight between about 3,000 g/mol and about 40,000 g/mol, between about 5,000 g/mol and about 30,000 g/mol, between about 6,000 g/mol and about 20,000 g/mol, between about 7,000 g/mol and about 12,000 g/mol, or between about 8,000 g/mol and about 10,000 g/mol. In some embodiments, the PEG is selected from the group consisting of PEG 3000, PEG 4000, PEG 5000, PEG 6000, PEG 7000, PEG 8000, PEG 9000, PEG 10000, and PEG 20000. In some embodiments, the loading solution comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM of PEG. The concentration of PEG in the loading buffer can be optimized based on the size of the molecule of interest, and/or the molecular weight of PEG. For example, a higher concentration of PEG is required for a smaller size of the molecules to be loaded or smaller molecular weight of PEG. Generally, the length of DNA of interest can be tuned by the PEG concentration and/or the PEG molecular weight. Larger PEGs can effectively condense longer DNA fragments at lower PEG concentration, while shorter fragments need higher concentration of the same molecular weight PEG. Alternatively, smaller PEG are more effective at condensing smaller DNA fragments at the same PEG concentration. In some embodiments, the loading solution comprises PEG 8000, for example, at a concentration of 2.5-25 mM or 5-15 mM.

In some embodiments, the loading solution further comprises one or more cations. The cation can be, e.g., a monovalent or divalent cation. In some embodiments, the loading solution comprises a monovalent cation, e.g., at a concentration of 50 to 500 mM or 100 to 300 mM, e.g., Na⁺ or K⁺. In some embodiments, the loading solution comprises a divalent cation, e.g., at a concentration of 0.05 to 10 mM, e.g., Sr²⁺. Combinations of cations can also be employed, e.g., K⁺ and Sr²⁺. In one exemplary class of embodiments, the solution comprises PEG 8000 and K⁺, e.g., 5-15 mM PEG 8000 and 100-300 mM K⁺. In one exemplary class of embodiments, the solution comprises PEG 8000, K⁺, and Sr²⁺, e.g., 5-15 mM PEG 8000, 100-300 mM K⁺, and 0.05-0.3 mM Sr²⁺.

In some examples, the loading solution comprises KOAc, SrOAc, or a combination thereof. In some examples, KOAc is at a concentration of about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM in the loading solution. In some examples, SrOAc is at a concentration of about 10 μM, about 20 μM, about 30 μM, about 40 μM, about 50 μM, about 60 μM, about 70 μM, about 80 μM, about 90 μM, about 100 μM, about 110 μM, about 120 μM, about 130 μM, about 140 μM, about 150 μM, about 160 μM, about 170 μM, about 180 μM, about 190 μM, about 200 μM, about 210 μM, about 220 μM, about 230 μM, about 240 μM, about 250 μM, about 260 μM, about 270 μM, about 280 μM, about 290 μM, or about 300 μM in the loading solution.

Provision of the PEG and cation can facilitate loading of large nucleic acids into the array regions. Thus, in some embodiments, the nucleic acids (e.g., the templates of the polymerase-template complexes) are at least about 250 bp in length, at least about 500 bp in length, at least about 1 kb in length, at least about 5 kb in length, at least about 10 kb in length, at least about 20 kb in length, at least about 30 kb in length, or at least about 40 kb in length. In general, the methods and compositions disclosed herein can be applied for loading nucleic acids between about 250 bp to about 40 kb in length. In a related exemplary class of embodiments, the templates of the polymerase-template complexes each comprise a double-stranded central region and two different single-stranded hairpin end regions. In another exemplary class of embodiments, polymerase-template complexes are distributed to the array regions, and the templates of the polymerase-template complexes comprise nicked or gapped double-stranded circular DNA molecules. In another exemplary class of embodiments, polymerase-template complexes are distributed to the array regions, and the templates of the polymerase-template complexes comprise linear molecules, e.g., double-stranded molecules, e.g., genomic DNA fragments or amplicons.

Loading nucleic acids into array regions can facilitate subsequent analysis of the nucleic acids, for example, nucleic acid sequencing, and in particular single-molecule nucleic acid sequencing. Thus, the methods optionally include analyzing the nucleic acids in the array regions, e.g., by determining their nucleic acid sequence. The PEG is optionally removed from the nucleic acids, e.g., by washing, prior to such analysis.

In some embodiments, the loading solution comprises about 0.1% (v/v), about 0.5% (v/v), about 1% (v/v), about 1.5% (v/v), about 2% (v/v), about 2.5% (v/v), about 3% (v/v), about 3.5% (v/v), about 4% (v/v), about 4.5% (v/v), about 5% (v/v), about 5.5% (v/v), about 6% (v/v), about 6.5% (v/v), about 7% (v/v), about 7.5% (v/v), about 8% (v/v), about 8.5% (v/v), about 9% (v/v), about 9.5% (v/v), or about 10% (v/v) of the viscosity adjusting agent. In some embodiments, the viscosity adjusting agent is selected from the group consisting of glycerol, a low molecular weight PEG, a polysaccharide such as cellulose, agar, dextrin, or trehalose, or polyvinylpyrrolidone (PVP).

Adding compatible surfactants and/or suitable viscosity adjusting agents to the sample mix solution and/or loading solution improves wetting while maintaining loading efficiency and sequencing performance. Surfactants having a lower surface tension and HLB value improve surface wetting in addition to other interactions with the ZMW array surface. Without wishing to be bound by theory, certain surfactants such as Tergitol contain a PEG moiety that may interact with DNA during the PEG-mediated condensation of sample to the ZMW array surface. These characteristics mitigate the observed effects on SNR by ensuring air is not partially filling the ZMW volume which reduces the sequencing mix volume and background fluorescence signal. The high concentration of surfactants may also contribute to passivating the ZMW array surface thereby aiding loading and protecting the polymerase-DNA complex from surface driven effects on sequencing processivity. The lower HLB value improves the interaction with the hydrophobic surface of the ZMW array which may improve the sample's interaction with the surface during loading and sequencing resulting in improved loading efficiency and sequencing rate. As a result, the ZMW array surface can be wetted directly with sample while maintaining sequencing base rate (bp/s) and photonic properties (SNR vs PkMid).

The disclosed technology employs wetting with sample directly to ensure direct contact of the sample with the ZMW array surface. While not wishing to be bound by theory, the loading efficiency increases due to the reduction in diffusion distance for the polymerase enzyme complexes to the ZMW surface. The viscosity adjusting agent and/or crowding agent aid in the delivery of the small volume to the ZMW surface by minimizing dilution and mixing between the complexes and the loading buffer.

In certain aspects, this disclosure also encompasses a loading solution comprising one or more surfactants such as nonionic surfactants as disclosed herein, and one or more crowding agents such as a high molecular weight PEG and polyvinylpyrrolidone (PVP). In some embodiments, the loading solution further comprises one or more polymerase enzyme complexes comprising a template nucleic acid and a polymerase enzyme, one or more viscosity adjusting agent, one or more salts (such as KOAc and/or SrOAc), or a combination thereof as disclosed herein.

In certain aspects, after distribution and optional immobilization of the nucleic acids (e.g., polymerase-template complexes), at least 38% of the array regions are occupied by a single immobilized nucleic acid (e.g., a single immobilized polymerase-template complex), e.g., at least 50% or at least 75% of the regions. In one exemplary class of embodiments, polymerase-template complexes are distributed to and immobilized nanoscale wells, and after the immobilizing step at least 38% or at least 50% of the nanoscale wells are occupied by a single immobilized polymerase-template complex.

The term “about” as used throughout the disclosure indicates the value of a given quantity varies by +/−10% of the value, or optionally +/−5% of the value, or in sone embodiments, by 1% of the value so disclosed. The term “about” also includes hie exact value “X” in addition to minor increments of “X” such as “X+0.1” or “X−0.1”.

In the following description, numerous specific details are set forth to provide a more thorough understanding of this disclosure. However, it will be apparent to one of skill in the art that this disclosure may be practiced without one or more of these specific details. In other instances, well-known features and procedures well known to those skilled in the art have not been described in order to avoid obscuring the invention.

### III. Compositions

The methods disclosed herein include processes for ensuring that a plurality of reaction sites in an array are occupied by a molecule of interest. In certain aspects, the array is an array of nanoscale wells, and the molecule of interest includes a complex of a polymerase enzyme and a template nucleic acid, where the template nucleic acid is in some aspects hybridized to a primer. Such complexes are able to under certain conditions generate signals that can be detected during performing the methods disclosed herein—often, those signals are generated by a nucleotide or a nucleotide analog that is labeled or otherwise detectable associating with its cognate base on the template nucleic acid and/or being incorporated. The following sections provide details on different types of compositions and components of use in the methods disclosed herein, including elements of the complexes that can be loaded into nanoscale wells. As will be appreciated, any of the compositions disclosed herein can be used in any combination with each other and in any of the methods further detailed in this disclosure.

### A. Template Molecules

The nucleic acids employed in the practice of the disclosed methods can be fully or partially double-stranded or can be single-stranded. Suitable nucleic acids include, but are not limited to, SMRTbells™ (circular nucleic acids having a double-stranded central region and single-stranded hairpin ends), double-stranded circular DNA molecules (e.g., nicked or gapped double-stranded circular DNA molecules, e.g., nicked or gapped plasmids), and linear molecules (e.g., genomic DNA fragments). In one exemplary class of embodiments, polymerase-template complexes are distributed to the array regions, and the templates of the polymerase-template complexes each comprise a double-stranded central region and two identical single-stranded hairpin end regions. In another exemplary class of embodiments, polymerase-template complexes are distributed to the array regions, and the templates of the polymerase-template complexes each comprise a double-stranded central region and two single-stranded hairpin end regions that are different from each other.

Nucleic acids, including template nucleic acids, can be prepared using techniques well known in the art, from essentially any desired sample. For further discussion of circular templates, including, e.g., simple circles and SMRTbells™ (circular nucleic acids having a double-stranded central region and single-stranded hairpin ends), see, e.g., U.S. Pat. Nos. 8,236,499 and 8,153,375 and Travers et al. (2010) Nucl. Acids Res. 38(15):e159, each of which is incorporated herein by reference in its entirety for all purposes.

Any of the methods, compositions, systems, and complexes disclosed herein can include template nucleic acid molecules, often as part of the polymerase enzyme complexes disclosed herein. In general, a template nucleic acid is a molecule for which the complementary sequence is (or can be) synthesized in a polymerase reaction. As will be appreciated, template sequences can be of any length or structure. In some cases, the template nucleic acid is linear; in some cases, the template nucleic acid is circular. The template nucleic acid can be DNA, RNA, and/or a non-natural RNA or DNA analog. Any nucleic acid that is suitable for replication by a polymerase enzyme can be used as a template in the methods and systems disclosed herein.

In some embodiments, the nucleic acids used in methods and compositions of this disclosure comprise nucleic acids obtained from a sample. The sample may comprise any number of things, including, but not limited to, bodily fluids (including, but not limited to, blood, urine, serum, lymph, saliva, anal and vaginal secretions, perspiration and semen) and cells of virtually any organism, including, but not limited to, mammalian samples, e.g., human samples; environmental samples (including, but not limited to, air, agricultural, water and soil samples); biological warfare agent samples; research samples (e.g., in the case of nucleic acids, the sample may be the products of an amplification reaction, including both target and signal amplification, such as PCR amplification reactions; and purified samples, such as purified genomic DNA, RNA preparations, raw samples (bacteria, virus, genomic DNA, etc.). As will be appreciated by those in the art, virtually any experimental manipulation may have been done on the samples.

In some embodiments, nucleic acid molecules are obtained from a sample and fragmented for use in (or prior to use in) methods disclosed herein, e.g., as template nucleic acids. The fragments may be single or double stranded and may further be modified in accordance with any methods known in the art and disclosed herein. Nucleic acids may be generated by fragmenting source nucleic acids, such as genomic DNA, using any method known in the art. In one embodiment, shear forces during lysis and extraction of genomic DNA generate fragments in a desired range. Also encompassed by the present disclosure are methods of fragmentation utilizing restriction endonucleases or transposases.

As will be appreciated, the nucleic acids may be generated from a source nucleic acid, such as genomic DNA, by fragmentation to produce fragments of a specific size. The nucleic acids can be, for example, from about 300 to about 50,000 nucleotides in length, e.g., 500-20,000, 600-1000, 700-1000, 700-900, 700-800, 800-1000, 900-1000, 1500-2000, 1750-2000, 500-21000, 600-20000, 700-19000, 800-18000, 900-17000, 1000-16000, 1100-15000, 1200-14000, 1300-13000, 1400-12000, 1500-11000, 1600-10000, 1700-9000, 1800-8000, 1900-7000, 2000-6000, 2100-5000, 2200-4000, 2300-3000, 5000-20000, 10000-30000, 12000-28000, 14000-26000, 16000-24000, 18000-22000, or 19000-20000 nucleotides in length. In some embodiments, the nucleic acids are at least 5000, 10000, 15000, 20000, 25000, 30000, 40000, 50000, 60000, 70000, 80000, 90000, 100,000, 120,000, 130,000, 140,000, 150,000, 200,000, 500,000, or 1,000,000 nucleotides in length. In some embodiments, the nucleic acids are part of polymerase-template complexes. In some embodiments, the nucleic acid templates are themselves further hybridized to primers.

In some cases, the template sequence may be a linear single or double stranded nucleic acid sequence. In still other embodiments, the template may be provided as a circular or functionally circular construct that allows redundant processing of the same nucleic acid sequence by the synthesis complex. Use of such circular constructs has been disclosed in, e.g., U.S. Pat. No. 7,315,019 and US Patent Publ. No. 2009/0029385, and alternate functional circular constructs are also disclosed in US Patent Publ. No. 2009/0298075, the full disclosures of each of which are incorporated herein by reference in their entirety for all purposes and in particular for all teachings related to template nucleic acid constructs. Briefly, such alternate constructs include template sequences that possess a central double stranded portion that is linked at each end by an appropriate linking oligonucleotide, such as a hairpin loop segment (SMRTbells™). Such structures not only provide the ability to repeatedly replicate a single molecule (and thus sequence that molecule), but also provide for additional redundancy by replicating both the sense and antisense portions of the double stranded portion. In the context of sequencing applications, such redundant sequencing provides great advantages in terms of sequence accuracy.

In some aspects, the template nucleic acid used in the compositions disclosed herein includes: a double stranded nucleic acid segment having a first and second end; a first hairpin oligonucleotide connecting each strand of the single template nucleic acid at the first end; and a second hairpin oligonucleotide connecting each strand of the single template nucleic acid at the second end. In some embodiments, the first hairpin and second hairpin oligonucleotide are identical.

In other embodiments, the first hairpin and second hairpin oligonucleotides are not identical—in other words, the template nucleic acid, despite being an alternate circular construct, is nevertheless asymmetrical. In further embodiments, the first hairpin oligonucleotide includes a primer binding site whereas the second hairpin oligonucleotide includes a capture adapter (or vice versa). The capture adapter is generally of a sequence that can be used to enrich a population for the hairpins of choice—for example, in some embodiments, the capture adapter comprises a polyA sequence, thereby allowing capture using beads or column chromatography utilizing polyT sequences. In some embodiments, the capture adapter comprises at least one methoxy residue. In some embodiments, the capture adapter is complementary to an oligonucleotide attached to a bead, which can in further embodiments be a magnetic bead that can be used to enrich a population for template nucleic acids containing the capture adapter. In some embodiments in which the population of templates includes templates with different adapters or in which each template comprises a different adapter at each end, different beads can be used which contain oligonucleotides complementary to the different adapters. Thus, for templates with two different adapters, two different beads can be used. For populations containing a plurality of different adapters, a concomitant number of different types of beads can be used that are directed to those adapters. In other embodiments, the same bead can contain different oligonucleotides complementary to the different adapters in the population of templates, such that the same bead can capture different adapters (and their associated templates).

In some embodiments, the first or second hairpin comprises a self-primed adapter sequence in which the primer is part of the adapter. In such embodiments, an additional oligonucleotide primer is not needed to allow a polymerase molecule to begin replicating the template.

In some embodiments, the nucleic acid template contains only a single hairpin at one end or the other.

### B. Nucleotides and Nucleotide Analogs

Nucleotides of use in this disclosure include, e.g., naturally occurring nucleotides such as dATP, dCTP, dGTP, and dTTP. Various nucleotide analogs are also of use in this disclosure, as discussed in further detail below. The analogs are optionally detectably labeled.

In certain aspects, non-incorporatable nucleotide analogs can be used, particularly for methods that rely on monitoring loading by detecting signals generated by interactions between nucleotides and/or nucleotide analogs and the cognate base on a template nucleic acid where the nucleotide and/or nucleotide analog is not incorporated into a nascent strand. Suitable non-incorporatable analogs are known in the art. See, e.g., U.S. Pat. Nos. 8,252,911, 8,530,164, and 8,652,781, previously incorporated by reference, for exemplary nonhydrolyzable (and therefore non-incorporatable) analogs. Exemplary nonhydrolyzable/non-incorporatable nucleotide analogs include, but are not limited to, analogs in which the phosphoester linkage between the alpha and beta phosphate of a nucleoside polyphosphate is replaced with a nonhydrolyzable linkage. For example, the oxygen group between the alpha and beta phosphate groups can be replaced with a nonhydrolyzable linkage, such as an amino, alkyl (e.g., methyl), thio, or other linkage not hydrolyzed by polymerase activity.

In certain aspects, nucleotides that terminate extension (reversibly or essentially irreversibly) can be used. Suitable extension terminating nucleotides and analogs are known in the art and include, but are not limited to, dideoxynucleotide triphosphates (ddNTPs), 3′-blocked nucleotides (nucleotides or analogs without a free 3′-hydroxyl group), for example, 3′-O-azidomethyl dNTPs, 3′-O-amino dNTPs, 3′-O-allyl dNTPs, and 3′-O-methyl-dNTPs, and 3′-unblocked terminators. For discussion and examples of reversible terminators, see, e.g., U.S. Pat. No. 9,175,342 and Chen et al. (2013) “The history and advances of reversible terminators used in new generations of sequencing technology” Genomics Proteomics Bioinformatics 11:34-40, previously incorporated by reference.

In certain aspects herein, nucleotides and/or nucleotide analogs that can be incorporated into a nascent strand without blocking incorporation of subsequent nucleotides and/or nucleotide analogs can be used.

As discussed, various polymerases can incorporate one or more nucleotide analogs into a growing oligonucleotide chain. Upon incorporation, the nucleotide analog can leave a residue that is the same as or different than a natural nucleotide in the growing oligonucleotide (the polymerase can incorporate any non-standard moiety of the analog, or can cleave it off during incorporation into the oligonucleotide). A “nucleotide analog” herein is a compound, that, in a particular application, functions in a manner similar or analogous to a naturally occurring nucleoside triphosphate (a “nucleotide”), and does not otherwise denote any particular structure. A nucleotide analog is an analog other than a standard naturally occurring nucleotide, i.e., other than A, G, C, T, or U, though upon incorporation into the oligonucleotide, the resulting residue in the oligonucleotide can be the same as (or different from) an A, G, C, T, or U residue.

In one useful aspect of this disclosure, nucleotide analogs can be modified to achieve any desired properties. For example, various linkers or other substituents can be incorporated into analogs that have the effect of reducing branching fraction, improving processivity, or altering rates. Modifications to the analogs can include extending the phosphate chains, e.g., to include a tetra-, penta-, hexa- or hepta-phosphate group, and/or adding chemical linkers to extend the distance between the nucleotide base and the dye molecule, e.g., a fluorescent dye molecule. Substitution of one or more non-bridging oxygen in the polyphosphate, for example with S or BH₃, can change the polymerase reaction kinetics. Optionally, one or more, two or more, three or more, or four or more non-bridging oxygen atoms in the polyphosphate group of the analog has an S substituted for an O. While not being bound by theory, it is believed that the properties of the nucleotide, such as the metal chelation properties, electronegativity, or steric properties, can be altered by substitution of the non-bridging oxygen(s).

Many nucleotide analogs are available and can be incorporated by polymerases. These include analog structures with core similarity to naturally occurring nucleotides, such as those that comprise one or more substituent on a phosphate, sugar, or base moiety of the nucleoside or nucleotide relative to a naturally occurring nucleoside or nucleotide. In one embodiment, the nucleotide analog includes three phosphate containing groups; for example, the analog can be a labeled nucleoside triphosphate analog and/or an α-thiophosphate nucleotide analog having three phosphate groups. In one embodiment, a nucleotide analog can include one or more extra phosphate containing groups, relative to a nucleoside triphosphate. For example, a variety of nucleotide analogs that comprise, e.g., from 4-6 or more phosphates are disclosed in detail in US Patent Publ. No. 2007-0072196, incorporated herein by reference in its entirety for all purposes. Other exemplary useful analogs, including tetraphosphate and pentaphosphate analogs, are disclosed in U.S. Pat. No. 7,041,812, incorporated herein by reference in its entirety for all purposes.

For example, the analog can include a labeled compound of the formula:

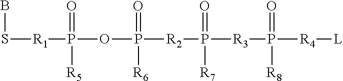

wherein B is a nucleobase (and optionally includes a label); S is selected from a sugar moiety, an acyclic moiety or a carbocyclic moiety (and optionally includes a label); L is an optional detectable label; R₁ is selected from O and S; R₂, R₃ and R₄ are independently selected from O, NH, S, methylene, substituted methylene, C(O), C(CH₂), CNH₂, CH₂CH₂, and C(OH)CH₂R where R is 4-pyridine or 1-imidazole, provided that R₄ may additionally be selected from

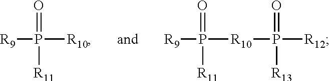

R₅, R₆, R₇, R₈, R₁₁ and R₁₃ are, when present, each independently selected from O, BH₃, and S; and R₉, R₁₀ and R₁₂ are independently selected from O, NH, S, methylene, substituted methylene, CNH₂, CH₂CH₂, and C(OH)CH₂R where R is 4-pyridine or 1-imidazole. In some cases, phosphonate analogs may be employed as the analogs, e.g., where one of R₂, R₃, R₄, R₉, R₁₀ or R₁₂ are not 0, e.g., they are methyl etc. See, e.g., US Patent Publ. No. 2007/0072196, previously incorporated herein by reference in its entirety for all purposes.

The base moiety incorporated into the analog is generally selected from any of the natural or non-natural nucleobases or nucleobase analogs, including, e.g., purine or pyrimidine bases that are routinely found in nucleic acids and available nucleic acid analogs, including adenine, thymine, guanine, cytosine, uracil, and in some cases, inosine. As noted, the base optionally includes a label moiety. For convenience, nucleotides and nucleotide analogs are generally referred to base upon their relative analogy to naturally occurring nucleotides. As such, an analog that operates, functionally, like adenosine triphosphate, may be generally referred to herein by the shorthand letter A. Likewise, the standard abbreviations of T, G, C, U and I, may be used in referring to analogs of naturally occurring nucleosides and nucleotides typically abbreviated in the same fashion. In some cases, a base may function in a more universal fashion, e.g., functioning like any of the purine bases in being able to hybridize with any pyrimidine base, or vice versa. The base moieties used in this disclosure may include the conventional bases disclosed herein or they may include such bases substituted at one or more side groups, or other fluorescent bases or base analogs, such as 1,N6 ethenoadenosine or pyrrolo C, in which an additional ring structure renders the B group neither a purine nor a pyrimidine. For example, in certain cases, it may be desirable to substitute one or more side groups of the base moiety with a labeling group or a component of a labeling group, such as one of a donor or acceptor fluorophore, or other labeling group. Examples of labeled nucleobases and processes for labeling such groups are disclosed in, e.g., U.S. Pat. Nos. 5,328,824 and 5,476,928, each of which is incorporated herein by reference in its entirety for all purposes.

In the analogs, the S group is optionally a sugar moiety that provides a suitable backbone for a synthesizing nucleic acid strand. For example, the sugar moiety is optionally selected from a D-ribosyl, 2′ or 3′ D-deoxyribosyl, 2′, 3′-D-dideoxyribosyl, 2′, 3′-D-didehydrodideoxyribosyl, 2′ or 3′ alkoxyribosyl, 2′ or 3′ aminoribosyl, 2′ or 3′ mercaptoribosyl, 2′ or 3′ alkothioribosyl, acyclic, carbocyclic or other modified sugar moieties. A variety of carbocyclic or acyclic moieties can be incorporated as the “S” group in place of a sugar moiety, including, e.g., those disclosed in US Patent Publ. No. 2003/0124576, which is incorporated herein by reference in its entirety for all purposes.

For most cases, the phosphorus containing chain in the analogs, e.g., a triphosphate in conventional NTPs, is preferably coupled to the 5′ hydroxyl group, as in natural nucleoside triphosphates. However, in some cases, the phosphorus containing chain is linked to the S group by the 3′ hydroxyl group.

L generally refers to a detectable labeling group that is coupled to the terminal phosphorus atom via the R₄ (or R₁₀ or R₁₂ etc.) group. The labeling groups employed in the analogs employed in this disclosure may comprise any of a variety of detectable labels. Detectable labels generally denote a chemical moiety that provides a basis for detection of the analog compound separate and apart from the same compound lacking such a labeling group. Examples of labels include, e.g., optical labels, e.g., labels that impart a detectable optical property to the analog, electrochemical labels, e.g., labels that impart a detectable electrical or electrochemical property to the analog, and physical labels, e.g., labels that impart a different physical or spatial property to the analog, e.g., a mass tag or molecular volume tag. In some cases individual labels or combinations may be used that impart more than one of the aforementioned properties to the analogs of this disclosure.

A variety of labels are known in the art and can be adapted to the practice of the methods disclosed herein. In one class of embodiments, the labels are optical labels, e.g., a fluorescent, a luminescent, a fluorogenic, a chemiluminescent, a chromophoric, or a chromogenic label, or another label that becomes detectable upon absorption of excitation radiation from an illumination source. Examples of preferred optically detectable labels include, e.g., organic fluorescent labels, such as cyanine-, fluorescein-, and/or rhodamine-based dyes, inorganic labels such as semiconductor nanocrystals, or quantum dots. In some embodiments, different labels share a fluorescent emission maximum but are nonetheless distinguishable by the amplitude of emission. Other examples of labels include particles that are optically detectable through their ability to scatter light. Such particles include any of the particle types disclosed elsewhere, herein, and particularly, metal nanoparticles, e.g., gold, silver, platinum, cobalt, or the like, which may be detected based upon a variety of different light scatter detection schemes, e.g., Rayleigh/Mie light scattering, surface enhanced Raman scattering, or the like. Other suitable labels include, but are not limited to, electrically detectable labels, enzymatically detectable labels, electrochemically detectable labels, and labels detectable based upon their mass. Mass labels include, e.g., particles or other large moieties that provide detectable variations in mass of the molecule to which they are attached or vary the molecule's rotational diffusion. Electrochemical labels that detectably alter the charge of the molecule, magnetic labels, such as magnetic particles, or the like can be employed. Other examples of suitable labels include groups that affect the flow of current, i.e., groups that alter (e.g., enhance or reduce) impedance or conductance of the composition. Such labels are useful, e.g., in applications where incorporation is detected by changes in conductance or impedance, e.g., in nanopore-based nucleic acid sequencing applications or nanoFET-based nucleic acid sequencing applications. Examples of conductance impacting functional groups include, e.g., long alkane chains which optionally include solubility enhancing groups, such as amido substitutions; long polyethylene glycol chains; polysaccharides; particles, such as latex, silica, polystyrene, metal, semiconductor, or dendrimeric particles; branched polymers, such as branched alkanes, branched polysaccharides, branched aryl chains; highly charged groups or polymers; oligopeptides; and oligonucleotides. Useful labels may additionally or alternatively include electrochemical groups that may be detected or otherwise exploited for their electrochemical properties, such as their overall electric charge. For example, highly charged groups can be included, like additional phosphate groups, sulfate groups, amino acid groups or chains, e.g., polylysine, polyarginine, etc. Likewise, redox active groups, such as redox active compounds, e.g., heme, or redox active enzymes, can be included. Other label types may include, e.g., magnetic particles that may be sensed through appropriate means, e.g., magneto-tunnel junction sensors, etc.

Optionally, the labeling groups incorporated into the analogs comprise optically detectable moieties, such as luminescent, chemiluminescent, fluorescent, fluorogenic, chromophoric and/or chromogenic moieties, with fluorescent and/or fluorogenic labels being preferred. A variety of different label moieties are readily employed in nucleotide analogs. Such groups include, e.g., fluorescein labels, rhodamine labels, cyanine labels (i.e., Cy3, Cy5, and the like, generally available from the Amersham Biosciences division of GE Healthcare), and the Alexa family of fluorescent dyes and other fluorescent and fluorogenic dyes available from Molecular Probes/Invitrogen, Inc. and disclosed in ‘The Handbook—A Guide to Fluorescent Probes and Labeling Technologies, Eleventh Edition’ (2010) (available from Invitrogen, Inc./Molecular Probes). A variety of other fluorescent and fluorogenic labels for use with nucleoside polyphosphates, and which would be applicable to the nucleotide analogs incorporated by polymerases, are disclosed in, e.g., US Patent Publ. No. 2003/0124576, previously incorporated herein by reference in its entirety for all purposes.

Thus, in one illustrative example, the analog can be a phosphate analog (e.g., an analog that has more than the typical number of phosphates found in nucleoside triphosphates) that includes, e.g., an Alexa dye label. For example, an Alexa488 dye can be labeled on a delta phosphate of a tetraphosphate analog (denoted, e.g., A488dC4P or A488dA4P, for the Alexa488 labeled tetraphosphate analogs of C and A, respectively), or an Alexa568 or Alexa633 dye can be used (e.g., A568dC4P and A633dC4P, respectively, for labeled tetraphosphate analogs of C or A568dT6P for a labeled hexaphosphate analog of T), or an Alexa546 dye can be used (e.g., A546dG4P), or an Alexa594 dye can be used (e.g., A594dT4P). As additional examples, an Alexa555 dye (e.g., A555dC6P or A555dA6P), an Alexa 647 dye (e.g., A647dG6P), an Alexa 568 dye (e.g., A568dT6P), and/or an Alexa660 dye (e.g., A660dA6P or A660dC6P) can be used in, e.g., single molecule sequencing. Similarly, to facilitate color separation, a pair of fluorophores exhibiting FRET (fluorescence resonance energy transfer) can be labeled on a delta phosphate of a tetraphosphate analog (denoted, e.g., FAM-amb-A532dG4P or FAM-amb-A594dT4P).

As disclosed herein, an analog can include a linker that extends the distance between the nucleotide base and the label moiety, e.g., a fluorescent dye moiety. Exemplary linkers and analogs are disclosed in U.S. Pat. No. 7,968,702. Similarly, a protein or other moiety can be employed to provide spacing and/or shielding between the base and the label, e.g., as disclosed in U.S. Pat. Nos. 9,062,091 and 9,957,291. Suitable polymerase substrates optionally include two or more nucleoside polyphosphates and/or two or more label moieties, e.g., as disclosed in U.S. Pat. Nos. 9,062,091 and 9,957,291 and US Patent Publ. No. 2009/0208957.

Additional details regarding labels, analogs, and methods of making such analogs can be found in US Patent Publ. Nos. 2007/0072196, 2009/0208957, 2010/0167299, 2010/0152424, 2017/0145495, 2017/0145496, and 2017/0321268; PCT Publ. Nos. WO 2007/041342 and WO 2009/114182; and U.S. Pat. Nos. 9,051,263, 8,669,374, 8,889,886, 8,906,612, 9,062,091, 9,957,291, each of which is incorporated herein by reference in its entirety for all purposes.

### C. Polymerases

The methods and compositions of the present disclosure utilize polymerase enzymes (also referred to herein as “polymerases”). Any suitable polymerase enzyme can be used in the systems and methods disclosed herein, particularly as part of the polymerase enzyme complexes loaded into reaction sites in accordance with this disclosure. Suitable polymerases include DNA dependent DNA polymerases, DNA dependent RNA polymerases, RNA dependent DNA polymerases (reverse transcriptases), and RNA dependent RNA polymerases. In certain embodiments, the polymerases used in the methods and compositions of this disclosure are strand-displacing polymerases.

As disclosed in further detail herein, polymerases of use in the presently disclosed methods can also include modifications that improve certain characteristics of the enzyme, including processivity, resistance to photodamage, and conduciveness to immobilization. In certain aspects, polymerases used in the methods and systems disclosed herein include a linker, motif (e.g., a biotin ligase recognition sequence), or domain through which the polymerases (and any other molecules they are complexed with, such as template nucleic acids and optionally replication initiating moieties) can be immobilized onto a surface e.g., through binding to a biotin-binding protein or other binding partner.

DNA polymerases are sometimes classified into six main groups based upon various phylogenetic relationships, e.g., with E. coli Pol I (class A), E. coli Pol II (class B), E. coli Pol III (class C), Euryarchaeotic Pol II (class D), human Pol beta (class X), and E. coli UmuC/DinB and eukaryotic RAD30/xeroderma pigmentosum variant (class Y). For a review of recent nomenclature, see, e.g., Burgers et al. (2001) “Eukaryotic DNA polymerases: proposal for a revised nomenclature” J Biol Chem. 276(47):43487-90. For a review of polymerases, see, e.g., Hubscher et al. (2002) “Eukaryotic DNA Polymerases” Annual Review of Biochemistry Vol. 71: 133-163; Alba (2001) “Protein Family Review: Replicative DNA Polymerases” Genome Biology 2(1): reviews 3002.1-3002.4; and Steitz (1999) “DNA polymerases: structural diversity and common mechanisms” J Biol Chem 274:17395-17398. The basic mechanisms of action for many polymerases have been determined. The three-dimensional structures of a large number of polymerases have been determined by x-ray crystallography and nuclear magnetic resonance (NMR) spectroscopy, including the structures of polymerases with bound templates, nucleotides, and/or nucleotide analogs. Many such structures are freely available for download from the Protein Data Bank, at (www(dot)rcsb(dot)org/pdb. Structures, along with domain and homology information, are also freely available for search and download from the National Center for Biotechnology Information's Molecular Modeling DataBase, at www(dot)ncbi(dot)nlm(dot)nih(dot)gov/Structure/MMDB/mmdb(dot)shtml. The structures of phi29 (Φ29) polymerase, Φ29 polymerase complexed with terminal protein, and Φ29 polymerase complexed with primer-template DNA in the presence and absence of a nucleoside triphosphate are available; see Kamtekar et al. (2004) “Insights into strand displacement and processivity from the crystal structure of the protein-primed DNA polymerase of bacteriophage Φ29” Mol. Cell 16(4): 609-618), Kamtekar et al. (2006) “The phi29 DNA polymerase:protein-primer structure suggests a model for the initiation to elongation transition” EMBO J. 25(6):1335-43, and Berman et al. (2007) “Structures of phi29 DNA polymerase complexed with substrate: The mechanism of translocation in B-family polymerases” EMBO J. 26:3494-3505, respectively. The structures of additional polymerases or complexes can be modeled, for example, based on homology of the polymerases with polymerases whose structures have already been determined. Alternatively, the structure of a given polymerase (e.g., a wild-type or modified polymerase), optionally complexed with a DNA or RNA (e.g., template and/or primer) and/or nucleotide analog, or the like, can be determined. information on structure determination and modeling is widely available in the art; see, e.g., U.S. Pat. No. 9,399,766 and references therein.

In addition to wild-type polymerases, chimeric polymerases made from a mosaic of different sources can be used. For example, Φ29 polymerases made by taking sequences from more than one parental polymerase into account can be used in methods disclosed herein. Chimeras can be produced, e.g., using consideration of similarity regions between the polymerases to define consensus sequences that are used in the chimera, or using gene shuffling technologies in which multiple Φ29-related polymerases are randomly or semi-randomly shuffled via available gene shuffling techniques (e.g., via “family gene shuffling”; see Crameri et al. (1998) “DNA shuffling of a family of genes from diverse species accelerates directed evolution” Nature 391:288-291; Clackson et al. (1991) “Making antibody fragments using phage display libraries” Nature 352:624-628; Gibbs et al. (2001) “Degenerate oligonucleotide gene shuffling (DOGS): a method for enhancing the frequency of recombination with family shuffling” Gene 271:13-20; and Hiraga and Arnold (2003) “General method for sequence-independent site-directed chimeragenesis: J. Mol. Biol. 330:287-296). In these methods, the recombination points can be predetermined such that the gene fragments assemble in the correct order. However, the combinations, e.g., chimeras, can be formed at random. For example, using methods disclosed in Clarkson et al., five gene chimeras, e.g., comprising segments of a Φ29 polymerase, a PZA polymerase, an M2 polymerase, a B103 polymerase, and a GA-1 polymerase, can be generated. Appropriate mutations to improve branching fraction, increase closed complex stability, or alter reaction rate constants can be introduced into the chimeras.

Available DNA polymerase enzymes have also been modified in any of a variety of ways, e.g., to reduce or eliminate exonuclease activities (many native DNA polymerases have a proof-reading exonuclease function that interferes with, e.g., sequencing applications), to simplify production by making protease digested enzyme fragments such as the Klenow fragment recombinant, etc. For example, polymerases have been modified to confer improvements in specificity, processivity, and improved retention time of labeled nucleotides in polymerase-DNA-nucleotide complexes (e.g., WO 2007/076057 Polymerases For Nucleotide Analogue Incorporation by Hanzel et al. and WO 2008/051530 Polymerase Enzymes And Reagents For Enhanced Nucleic Acid Sequencing by Rank et al.), to alter branch fraction and translocation (e.g., US Pub. No. 20100075332 entitled “Engineering Polymerases And Reaction Conditions For Modified Incorporation Properties”), to increase photostability (e.g., US Pub. No. 20100093555 entitled “Enzymes Resistant to Photodamage”), and to improve surface-immobilized enzyme activities (e.g., WO 2007/075987 Active Surface Coupled Polymerases by Hanzel et al. and WO 2007/076057 Protein Engineering Strategies To Optimize Activity Of Surface Attached Proteins by Hanzel et al.). In some cases, the polymerase is modified in order to more effectively incorporate desired nucleotide analogs, e.g. analogs having four or more phosphates in their polyphosphate chain. Enzymes mutated to more readily accept nucleotide analogs having such properties are disclosed, for example in the applications disclosed herein and in US 20120034602-Recombinant Polymerases for Improved Single Molecule Sequencing; US 20100093555-Enzymes Resistant to Photodamage; US 20110189659-Generation of Modified Polymerases for Improved Accuracy in Single Molecule Sequencing; US 20100112645-Generation of Modified Polymerases for Improved Accuracy in Single Molecule Sequencing; US 2008/0108082-Polymerase enzymes and reagents for enhanced nucleic acid sequencing; and US 20110059505-Polymerases for Nucleotide Analogue Incorporation. Each of these references is incorporated herein by reference in its entirety for all purposes.

Many polymerases that are suitable, e.g., for use in sequencing, labeling and amplification technologies, are available. For example, human DNA Polymerase Beta is available from R&D systems. DNA polymerase I is available from Epicenter, GE Health Care, Invitrogen, New England Biolabs, Promega, Roche Applied Science, Sigma Aldrich and many others. The Klenow fragment of DNA Polymerase I is available in both recombinant and protease digested versions, from, e.g., Ambion, Chimerx, eEnzyme LLC, GE Health Care, Invitrogen, New England Biolabs, Promega, Roche Applied Science, Sigma Aldrich and many others. Φ29 DNA polymerase is available from e.g., Epicentre. Poly A polymerase, reverse transcriptase, Sequenase, SP6 DNA polymerase, T4 DNA polymerase, T7 DNA polymerase, and a variety of thermostable DNA polymerases (Taq, hot start, titanium Taq, etc.) are available from a variety of these and other sources. Recent commercial DNA polymerases include Phusion™ High-Fidelity DNA Polymerase, available from New England Biolabs; GoTaq® Flexi DNA Polymerase, available from Promega; RepliPHI™ Φ29 DNA Polymerase, available from Epicentre Biotechnologies; PfuUltra™ Hotstart DNA Polymerase, available from Stratagene; KOD HiFi DNA Polymerase, available from Novagen; and many others. Biocompare(dot)com provides comparisons of many different commercially available polymerases.

DNA polymerases that can be employed, e.g., in single molecule sequencing or other techniques of use with methods and compositions of this disclosure, include, e.g., Taq polymerases, exonuclease deficient Taq polymerases, E. coli DNA Polymerase 1, Klenow fragment, reverse transcriptases, Φ29-related polymerases including wild type Φ29 polymerase and derivatives of such polymerases such as exonuclease deficient forms, T7 DNA polymerase, T5 DNA polymerase, an RB69 polymerase, etc.

In one aspect, the polymerase of use in the methods and compositions disclosed herein is a modified Φ29-type DNA polymerase. For example, the modified recombinant DNA polymerase can be homologous to a wild-type or exonuclease deficient Φ29 DNA polymerase, e.g., as disclosed in U.S. Pat. Nos. 5,001,050, 5,198,543, or 5,576,204. Alternately, the modified recombinant DNA polymerase can be homologous to other Φ29-type DNA polymerases, such as B103, GA-1, PZA, 015, BS32, M2Y, Nf, G1, Cp-1, PRD1, PZE, SFS, Cp-5, Cp-7, PR4, PR5, PR722, L17, Φ21, or the like. For nomenclature, see also, Meijer et al. (2001) “Φ29 Family of Phages” Microbiology and Molecular Biology Reviews, 65(2):261-287. Suitable polymerases (including polymerases with two biotinylation sites that constitute a bis-biotin tag) are disclosed, for example, in US Patent Publ. Nos. 2007/0196846, 2008/0108082, 2010/0075332, 2010/0093555, 2010/0112645, 2011/0189659, 2012/0034602, 2013/0217007, 2014/0094374, and 2014/0094375, each of which is incorporated herein by reference in its entirety for all purposes.

In some embodiments, the polymerase enzyme used in the methods disclosed herein includes RNA dependent DNA polymerases or reverse transcriptases. Suitable reverse transcriptase enzymes include HIV-1, M-MLV, AMV, and Telomere Reverse Transcriptase. Reverse transcriptases also allow for the direct sequencing of RNA substrates such as messenger RNA, transfer RNA, non-coding RNA, ribosomal RNA, micro RNA or catalytic RNA.

The polymerase enzymes of use in the methods and compositions disclosed herein generally require a primer. While in most cases an oligonucleotide primer is used, in some cases a protein such as a terminal protein can act as a primer. Oligonucleotide primers are generally complementary to a portion of the template nucleic acid. The primers can comprise naturally occurring RNA or DNA oligonucleotides. The primers may also be synthetic analogs. The primers may have alternative backbones as disclosed herein. The primers may also have other modifications, such as the inclusion of heteroatoms, the attachment of labels, such as dyes, or substitution with functional groups which will still allow for base pairing and for recognition by the enzyme. Primers can select tighter binding primer sequences, e.g., GC rich sequences, as well as employ primers that include within their structure non-natural nucleotides or nucleotide analogs, e.g., peptide nucleic acids (PNAs) or locked nucleic acids (LNAs), that can demonstrate higher affinity pairing with the template. The primers can also be selected to influence the kinetics of the polymerase reaction through the use of length, nucleotide content, and/or any of the modifications disclosed herein. In other embodiments, self-priming templates are employed. For example, a SMRTbell™ (circular nucleic acid having a double-stranded central region and single-stranded hairpin ends) including a self-primed adapter sequence can be employed, as disclosed herein. As another example, a double-stranded template including at least one nick or gap can be employed (e.g., a nicked or gapped double-stranded plasmid).

To reduce or prevent undesired dissociation of the polymerase from the template and primer, the processivity of the polymerase can be increased by locking the template in place in the enzyme, e.g., with chemical cross-links. For example, a bifunctional cross-linker can be reacted with residues in the polymerase on each side of the bound template, topologically encircling the template. See, e.g., U.S. Pat. No. 7,745,116 and US Patent Publ. No. 2015/0086994, each of which is incorporated herein by reference in its entirety for all purposes. Cysteine residues can be introduced into the polymerase at suitable positions for cross-link formation. For example, a recombinant Φ29 polymerase can include, e.g., A83C and E420C substitutions, D84C and E418C substitutions, V19C and N409C substitutions, and/or N409C and V568C substitutions. See, e.g., US Patent Publ. No 2014/0094375, incorporated herein by reference in its entirety for all purposes, for the sequence of wild-type Φ29 polymerase. Existing solvent accessible cysteine residues can be mutated to ensure that the cross-link is formed between the desired pair of residues; thus, a suitable recombinant Φ29 polymerase can also include one or more substitutions such as, e.g., C106S and/or C448V. Suitable bifunctional linkers are known in the art, for example, a bismaleimide linker, e.g., a bismaleimide-PEG linker, e.g., 1,11-bismaleimido-triethyleneglycol (BM(PEG)₃). Other coupling chemistries that can be employed include, e.g., thiol reactive reagents and disulfide containing reagents, e.g., haloacetyl crosslinkers (e.g., linkers including two iodoacetyl/iodoacetamide or bromoacetyl groups) and linkers with two pyridyl disulfide groups. The body of the linker can include, e.g., PEG (polyethylene glycol), an oligopeptide (e.g., polyglycine), or the like. Optimal linker length can be chosen based on the distance between the two residues to be cross-linked, e.g., in a crystal structure or other model of the polymerase. The linker is typically reacted with the polymerase after binding of the template (or primer/template); suitable reaction conditions for various linker chemistries are known in the art. Noncovalent linkers can also be employed. Such topological encirclement of the template by polymerase can be particularly effective for circular templates (including, e.g., simple circles and SMRTbells™ (circular nucleic acids having a double-stranded central region and single-stranded hairpin ends) as disclosed in, e.g., U.S. Pat. No. 8,153,375 and Travers et al. (2010) Nucl. Acids Res. 38(15):e159, each of which is incorporated herein by reference in its entirety for all purposes).

Many native DNA polymerases have a proof-reading exonuclease function which can yield substantial data analysis problems in processes that utilize real time observation of incorporation events as a method of identifying sequence information, e.g., single molecule sequencing applications. Even where exonuclease activity does not introduce such problems in single molecule sequencing, reduction of exonuclease activity can be desirable since it can increase accuracy (in some cases at the expense of read length).

Accordingly, polymerases for use in the techniques disclosed herein optionally include one or more mutations (e.g., substitutions, insertions, and/or deletions) relative to the parental polymerase that reduce or eliminate endogenous exonuclease activity. For example, relative to wild-type Φ29 DNA polymerase, one or more of positions N62, D12, E14, T15, H61, D66, D169, K143, Y148, and H149 is optionally mutated to reduce exonuclease activity in a recombinant Φ29 polymerase. Exemplary mutations that can reduce exonuclease activity in a recombinant Φ29 polymerase include, e.g., N62D, N62H, D12A, T151, E141, E14A, D66A, K143D, D145A and D169A substitutions, as well as addition of an exogenous feature at the C-terminus (e.g., a polyhistidine tag). See, e.g., US Patent Publ. No. 2014/0094375, incorporated herein by reference in its entirety for all purposes, for the sequence of wild-type Φ29 polymerase.

Additional examples of the polymerases for use in the techniques disclosed herein are disclosed in U.S. Pat. Nos. 8,323,939, 8,921,086, 8,343,746, 8,257,954, 8,999,676, 9,296,999, and 9,399,766, the contents of which are incorporated herein by reference in their entireties.

### IV. Substrates and Surfaces

Substrates of use in the methods disclosed herein are known in the art and discussed herein, and as will be appreciated, any of the substrates discussed herein can be used in any combination for any embodiments discussed herein.

In exemplary embodiments, the loading methods disclosed herein are generally used for loading molecules of interest, including polymerase enzyme complexes, onto substrates that include one or more reaction regions (also referred to herein as “array regions”) arranged in the form of an array on an inert substrate material, also referred to herein as a “solid support” or “surface,” that allows for combination of the reactants, e.g., in a sequencing reaction, in a defined space. Arrays can be regular or irregular, e.g., random. The substrates and array regions can also allow for detection, e.g., of the sequencing reaction event. As disclosed herein, nucleic acids or polymerase complexes can be deposited in the reaction regions such that individual nucleic acids (or polymerase reactions) are independently optically observable. A reaction region can be a localized area on the substrate material that facilitates interaction of reactants, e.g., in a nucleic acid sequencing reaction. A reaction region may in certain embodiments be a nanoscale well (also referred to herein as a nanowell), and in further embodiments the nanowell is a ZMW. A nanoscale well typically has dimensions in the nanometer range, i.e., less than 1 micrometer and more than 1 nanometer. In some embodiments, a nanoscale well has a cross-sectional diameter of less than 1000, 900, 800, 700, 600, or 500 nm, e.g., less than 400, 350, 300, 250, 200, 150, or 100 nm. In some embodiments, a nanoscale well has a cross-sectional diameter of at least 50 nm, such as 50-150 nm or 80-100 nm. In some embodiments, a nanoscale well has a depth of less than 1000, 900, 800, 700, 600, or 500 nm, e.g., less than 400, 350, 300, 250, or 200 nm. In some embodiments, a nanoscale well has a depth of at least 100, 150, or 200 nm. In some embodiments, a nanoscale well has a depth of between 100 and 1000 nm. Preferably, a nanoscale well has a depth of between 200 and 600 nm. As discussed herein, the loading and then subsequent sequencing reactions contemplated by the disclosure can in some embodiments occur on numerous individual nucleic acid samples in tandem, in particular simultaneously sequencing numerous nucleic acid samples, e.g., derived from genomic and chromosomal DNA. The apparatus of this disclosure can therefore include an array having a sufficient number of array regions/reaction regions to carry out such numerous individual sequencing reactions. In one embodiment, the array comprises at least 1,000 reaction regions. In another embodiment, the array comprises greater than 400,000 reaction regions, preferably between 400,000 and 20,000,000 reaction regions. In a more preferred embodiment, the array comprises between 1,000,000 and 16,000,000 reaction regions, e.g., 1,000,000, 2,000,000, 3,000,000, 4,000,000, 5,000,000, 6,000,000, 7,000,000, 8,000,000, 9,000,000, or 10,000,000 reaction regions.

The reaction regions on the array may take the form of a cavity or well in the substrate material, having a width and depth, into which reactants can be deposited. One or more of the reactants typically are bound to the substrate material in the reaction region and the remainder of the reactants are in a medium which facilitates the reaction and which flows through or contacts the reaction region. When formed as cavities or wells, the chambers are preferably of sufficient dimension to allow for (i) the introduction of the necessary reactants into the chambers, (ii) reactions to take place within the chamber, and (iii) inhibition of mixing of reactants between chambers. The shape of the well or cavity is preferably circular or cylindrical, but can be multisided so as to approximate a circular or cylindrical shape. In another embodiment, the shape of the well or cavity is substantially hexagonal. The cavity can have a smooth wall surface. In an additional embodiment, the cavity can have at least one irregular wall surface. The cavities can have, e.g., a planar bottom or a concave bottom.

The reaction regions may in some situations take the form of a nanopore. Such reaction regions, including arrays of nanopores, are known in the art and disclosed for example in US Published App. Nos. 2013/0327644 and 2014/0051068, which are hereby incorporated by reference in their entirety for all purposes and in particular for all teachings related to nanopore arrays.

In general, the reaction regions into which molecules of interest are loaded in accordance with the methods disclosed herein are of a configuration that any signals generated by the molecules of interest are only detectable when those molecules are within the reaction region, e.g., within the nanoscale well (e.g., in an observation volume in the well), within or proximal to the nanopores, or attached to the gate of a nanoFET.

Any material can be used as the solid support material, as long as the surface allows for stable attachment of polymerase enzyme complexes and optionally detection of nucleotide incorporation. The solid support material can be, e.g., planar or cavitated, e.g., in a cavitated terminus of a fiber optic or in a microwell etched, molded, or otherwise micromachined into the planar surface, e.g. using techniques commonly used in the construction of microelectromechanical systems. See e.g., Rai-Choudhury, HANDBOOK OF MICROLITHOGRAPHY, MICROMACHINING, AND MICROFABRICATION, VOLUME 1: MICROLITHOGRAPHY, Volume PM39, SPIE Press (1997); Madou, CRC Press (1997), Aoki, Biotech. Histochem. 67: 98-9 (1992); Kane et al., Biomaterials. 20: 2363-76 (1999); Deng et al., Anal. Chem. 72:3176-80 (2000); Zhu et al., Nat. Genet. 26:283-9 (2000). In some embodiments, the solid support is optically transparent, e.g., glass, silicon oxide and silicon dioxide. In some embodiments, the surface of the solid support or the substrate is functionalized. Methods of functionalizing a surface of a substrate are disclosed, for example in U.S. Pat. No. 8,501,406, which is incorporated herein by reference in its entireties for all purposes and in particular for all teachings related to surface functionalization.

Suitable substrates include chips having arrays of nanoscale wells or ZMWs. Exemplary substrates include substrates having a metal or metal oxide layer on a silica-based layer, with nanoscale wells disposed through the metal or metal layer to or into the silica-based layer. Such substrates are disclosed, for example in U.S. patent application Ser. Nos. 10/259,268, 14/187,198, 14/107,730, and 13/920,037, and U.S. Pat. Nos. 8,994,946, 8,906,670, 8,993,307, 8,802,600, 7,907,800, and 7,302,146, which are incorporated herein by reference in their entireties for all purposes and in particular for all teachings related to substrates. Biotinylation of such substrates is disclosed, e.g., in U.S. Pat. Nos. 7,763,423 and 8,802,600 and US Patent Publ. No. 2017-0184580 (which are incorporated herein by reference in their entireties for all purposes), as is loading and immobilization of nucleic acids, polymerases, and other molecules on such substrates. Other suitable substrates include, but are not limited to, chips having arrays of nanopores, chips having arrays of wells or apertures that comprise a bilayer in which one or more nanopores are inserted, and chips having arrays of nanoFETS.

### V. Applications for Disclosed Methods and Compositions: Sequencing

The methods, devices, systems, and compositions disclosed herein are particularly useful for loading arrays that can then be used, e.g., in single molecule sequencing methods, and specifically single molecule sequencing by incorporation in real time or by nanopore sequencing, because the methods and compositions of the present disclosure provide a way to load reaction regions with a reduced amount of a composition such as a nucleic acid or a reaction complex that includes a polymerase complexed to a template nucleic acid. In general, the loading achieved by methods and compositions disclosed herein allow single molecule analysis to be conducted more efficiently because the required sample input is reduced by 2- to 10-fold.

Once the nanoscale wells are occupied by polymerase enzyme complexes after loading, the array can then be washed and further processed to prepare the array for downstream applications, such as sequencing reactions. For use in sequencing-by-synthesis reactions, the wash steps can include washing with buffers to remove any metal ions maintaining the polymerases in an inactive state, unincorporatable nucleotide analogs, additives that slow the polymerase, and/or like reagents employed during loading, thus allowing incorporation of nucleotides and nucleotide analogs and the generation of sequencing signals as the polymerases form nascent strands. The wash and further processing steps can include any steps useful for any of the sequencing reactions disclosed herein and known in the art. In certain exemplary embodiments, the sequencing reactions include the steps of providing one or more nucleotides or nucleotide analogs (e.g., labeled analogs); performing a polymerization reaction in which the polymerase enzyme replicates at least a portion of the template nucleic acid in a template-dependent 43 manner, whereby one or more of the nucleotides or nucleotide analogs are incorporated into the resulting nucleic acid; and identifying a time sequence of incorporation of the one or more nucleotide or nucleotide analogs into the resulting nucleic acid.

In some aspects, this disclosure includes methods of analyzing the sequence of template nucleic acids. In such aspects, the sequence analysis optionally employs template dependent synthesis in identifying the nucleotide sequence of the template nucleic acid. Nucleic acid sequence analysis that employs template dependent synthesis identifies individual bases, or groups of bases, as they are added during a template mediated synthesis reaction, such as a primer extension reaction, where the identity of the base is required to be complementary to the template sequence to which the primer sequence is hybridized during synthesis. Other such processes include ligation driven processes, where oligo- or polynucleotides are complexed with an underlying template sequence, in order to identify the sequence of nucleotides in that sequence. Typically, such processes are enzymatically mediated using nucleic acid polymerases, such as DNA polymerases, RNA polymerases, reverse transcriptases, and the like, or other enzymes such as in the case of ligation driven processes, e.g., ligases.

Sequence analysis using template dependent synthesis can include a number of different processes. For example, in embodiments utilizing sequence by synthesis processes, individual nucleotides or nucleotide analogs are identified iteratively as they are added to the growing primer extension product.

In some aspects, the methods disclosed herein include steps from any single molecule sequencing methods known in the art. See, e.g., Rigler, et al., DNA-Sequencing at the Single Molecule Level, Journal of Biotechnology, 86(3): 161 (2001); Goodwin, P. M., et al., Application of Single Molecule Detection to DNA Sequencing. Nucleosides & Nucleotides, 16(5-6): 543-550 (1997); Howorka, S., et al., Sequence-Specific Detection of Individual DNA Strands using Engineered Nanopores, Nature Biotechnology, 19(7): 636-639 (2001); Meller, A., et al., Rapid Nanopore Discrimination Between Single Polynucleotide Molecules, Proceedings of the National Academy of Sciences of the United States of America, 97(3): 1079-1084 (2000); Driscoll, R. J., et al., Atomic-Scale Imaging of DNA Using Scanning Tunneling Microscopy. Nature, 346(6281): 294-296 (1990).

In some embodiments, methods of single molecule sequencing known in the art include detecting individual nucleotides as they are incorporated into a primed template, i.e., sequencing by synthesis. Such methods can utilize exonucleases to sequentially release individual fluorescently labeled bases as a second step after DNA polymerase has formed a complete complementary strand. See Goodwin et al., “Application of Single Molecule Detection to DNA Sequencing,” Nucleos. Nucleot. 16: 543-550 (1997).

In general, for sequencing methods utilizing compositions disclosed herein, individual polymerase compositions are provided within separate discrete regions of a support. For example, in some cases, individual complexes may be provided within individual confinement structures, including nanoscale structures such as nanoscale wells. In some examples, zero-mode waveguide cores or any of the reaction regions discussed herein in the stepwise sequencing section serve as the reaction regions for sequencing methods utilizing compositions disclosed herein. Examples of waveguides and processes for immobilizing individual complexes therein are disclosed in, e.g., PCT Publ. No. WO 2007/123763, the full disclosure of which is incorporated herein by reference in its entirety for all purposes and in particular for all teachings related to providing individual complexes into individual confinement structures. In some cases the molecules of interest (e.g., polymerase/template complexes) can be provided onto or proximal to structures or regions that allow for electronic single molecule sequencing. Such structures can include nanoscale electronic structures such as electrodes, capacitors, or field effect transducers (nanoFETs). NanoFETs include those having carbon nanotube gates. Such structures and their use for single molecule sequencing are disclosed, for example, in US Patent Publ. Nos. 2015/0065353 and 2017/0037462, which are incorporated herein in their entirety for all purposes and in particular for all teachings related to structures for use in single molecule sequencing.

In one example reaction of interest, a polymerase reaction can be isolated within an extremely small observation volume that effectively results in observation of individual polymerase molecules. As a result, the incorporation event provides observation of an incorporating nucleotide analog that is readily distinguishable from non-incorporated nucleotide analogs. In a preferred aspect, such small observation volumes are provided by immobilizing the polymerase enzyme within an optical confinement, such as a Zero Mode Waveguide (ZMW). For a description of ZMWs and their application in single molecule analyses, and particularly nucleic acid sequencing, see, e.g., US Patent Publ. No. 2003/0044781 and U.S. Pat. No. 6,917,726, each of which is incorporated herein by reference in its entirety for all purposes. See also Levene et al. (2003) “Zero-mode waveguides for single-molecule analysis at high concentrations” Science 299:682-686, Eid et al. (2009) “Real-time DNA sequencing from single polymerase molecules” Science 323:133-138, and U.S. Pat. Nos. 7,056,676, 7,056,661, 7,052,847, and 7,033,764, the full disclosures of which are incorporated herein by reference in their entirety for all purposes.

In general, a polymerase enzyme is complexed with the template strand in the presence of one or more nucleotides and/or one or more nucleotide analogs. For example, in certain embodiments, labeled analogs are present representing analogous compounds to each of the four natural nucleotides, A, T, G and C, e.g., in separate polymerase reactions, as in classical Sanger sequencing, or multiplexed together, e.g., in a single reaction, as in multiplexed sequencing approaches. When a particular base in the template strand is encountered by the polymerase during the polymerization reaction, it complexes with an available analog that is complementary to such nucleotide, and incorporates that analog into the nascent and growing nucleic acid strand. In one aspect, incorporation can result in a label being released, e.g., in polyphosphate analogs, cleaving between the a and R phosphorus atoms in the analog, and consequently releasing the labeling group (or a portion thereof). The incorporation event is detected, either by virtue of a longer presence of the analog and, thus, the label, in the complex, or by virtue of release of the label group into the surrounding medium. Where different labeling groups are used for each of the types of analogs, e.g., A, T, G or C, identification of a label of an incorporated analog allows identification of that analog and consequently, determination of the complementary nucleotide in the template strand being processed at that time. Sequential reaction and monitoring permits real-time monitoring of the polymerization reaction and determination of the sequence of the template nucleic acid. As disclosed herein, in particularly preferred aspects, the polymerase enzyme/template complex is provided immobilized within an optical confinement that permits observation of an individual complex, e.g., a zero-mode waveguide. For additional information on single molecule sequencing monitoring incorporation of phosphate-labeled analogs in real time, see, e.g., Eid et al. (2009) “Real-time DNA sequencing from single polymerase molecules” Science 323:133-138.

In a first exemplary technique, a nucleic acid synthesis complex including a polymerase enzyme, a template sequence and a complementary primer sequence is provided immobilized within an observation region that permits illumination and observation of a small volume that includes the complex without excessive illumination of the surrounding volume. By illuminating and observing only the volume immediately surrounding the complex, one can readily identify fluorescently labeled nucleotides that become incorporated during that synthesis, as such nucleotides are retained within that observation volume by the polymerase for longer periods than those nucleotides that are simply randomly diffusing into and out of that volume. In particular, when a nucleotide is incorporated into DNA by the polymerase, it is retained within the observation volume for a prolonged period of time, and upon continued illumination yields a prolonged fluorescent signal. By comparison, randomly diffusing and not incorporated nucleotides remain within the observation volume for much shorter periods of time, and thus produce only transient signals, many of which go undetected due to their extremely short duration.

In particularly preferred exemplary systems, the confined illumination volume is provided through the use of arrays of optically confined apertures termed zero mode waveguides (ZMWs). See, e.g., U.S. Pat. No. 6,917,726, which is incorporated herein by reference in its entirety for all purposes. For sequencing applications, the DNA polymerase is typically provided immobilized upon the bottom of the ZMW, although another component of the complex (e.g., a primer or template) is optionally immobilized on the bottom of the ZMW to localize the complex. See, e.g., Korlach et al. (2008) Proc Natl Acad Sci USA 105(4):1176-1181 and US Patent Publ. No. 2008/0032301, each of which is incorporated herein by reference in its entirety for all purposes.

In operation, the fluorescently labeled nucleotides (e.g., analogs corresponding to A, C, G and T) bear one or more fluorescent dye groups on a terminal phosphate moiety that is cleaved from the nucleotide upon incorporation. As a result, synthesized nucleic acids do not bear the build-up of fluorescent labels, as the labeled polyphosphate groups diffuse away from the complex following incorporation of the associated nucleotide, nor do such labels interfere with the incorporation event. See, e.g., Korlach et al. (2008) Nucleosides, Nucleotides and Nucleic Acids 27:1072-1083.

In a second exemplary technique, the immobilized complex and the nucleotides to be incorporated are each provided with interactive labeling components. Upon incorporation, the nucleotide borne labeling component is brought into sufficient proximity to the complex borne (or complex proximal) labeling component, such that these components produce a characteristic signal event. For example, the polymerase may be provided with a fluorophore that provides fluorescent resonant energy transfer (FRET) to appropriate acceptor fluorophores. These acceptor fluorophores are provided upon the nucleotide to be incorporated, where each type of nucleotide bears a different acceptor fluorophore, e.g., that provides a different fluorescent signal. Upon incorporation, the donor and acceptor are brought close enough together to generate energy transfer signal. By providing different acceptor labels on the different types of nucleotides, one obtains a characteristic FRET-based fluorescent signal for the incorporation of each type of nucleotide, as the incorporation is occurring.

In a related aspect, a nucleotide analog may include two interacting fluorophores that operate as a donor/quencher pair, where one member is present on the nucleobase or other retained portion of the nucleotide, while the other member is present on a phosphate group or other portion of the nucleotide that is released upon incorporation, e.g., a terminal phosphate group. Prior to incorporation, the donor and quencher are sufficiently proximal on the same analog as to provide characteristic signal quenching. Upon incorporation and cleavage of the terminal phosphate groups, e.g., bearing a donor fluorophore, the quenching is removed and the resulting characteristic fluorescent signal of the donor is observable.

The sequencing processes, e.g., using the substrates disclosed herein and the compositions disclosed herein, are generally exploited in the context of a fluorescence optical system that is capable of illuminating the various complexes on the substrate, and obtaining, detecting and separately recording fluorescent signals from these complexes. Such systems typically employ one or more illumination sources that provide excitation light of appropriate wavelength(s) for the labels being used. An optical train directs the excitation light at the reaction region(s) and collects emitted fluorescent signals and directs them to an appropriate detector or detectors. Additional components of the optical train can provide for separation of spectrally different signals, e.g., from different fluorescent labels, and direction of these separated signals to different portions of a single detector or to different detectors. Other components may provide for spatial filtering of optical signals, focusing and direction of the excitation and/or emission light to and from the substrate. An exemplary system is also disclosed in US Patent Publ. No. 2007/0036511 and Lundquist et al. (2008) Optics Letters 33(9):1026-1028, the full disclosures of which are incorporated herein by reference in their entirety for all purposes. Fluorescence reflective optical trains can be used in the applications of the systems of this disclosure. For a discussion on the advantages of such systems, see, e.g., US Patent Publ. Nos. 2007/0188750 (“Methods and systems for simultaneous real-time monitoring of optical signals from multiple sources”), 2008/0030628 (“Methods and systems for simultaneous real-time monitoring of optical signals from multiple sources”), and 2007/0206187 (“Methods and systems for simultaneous real-time monitoring of optical signals from multiple sources”), the full disclosures of which are incorporated herein by reference in their entirety for all purposes.

In the context of the nucleic acid sequencing methods disclosed herein, it will be appreciated that the signal sources each represent sequencing reactions, and particularly, polymerase mediated, template dependent primer extension reactions, where in preferred aspects, each base incorporation event results in a prolonged illumination (or localization) of one of four differentially labeled nucleotides being incorporated, so as to yield a recognizable pulse (peak) that carries a distinguishable spectral profile and/or color. Similar reactions may also be used to detect the presence of polymerase enzyme complexes within the nanoscale wells in accordance with the loading methods disclosed herein.

In other embodiments, the reaction sites into which molecules of interest are loaded are nanopores. As will be appreciated, any of the loading methods disclosed herein with respect to loading of arrays of nanoscale wells applies equally to nanopores. In exemplary embodiments, polymerase enzyme complexes are loaded into a nanopore—the nanopore comprises binding moieties complementary to reaction moieties on the enzyme (or another molecule associated with the enzyme, e.g., a template). In this way, a single enzyme complex is loaded into each of a plurality of nanopores. In certain embodiments, the complexes are attached proximal to the nanopore. As will be appreciated, helicases, exonucleases, and/or other motor proteins can be used in addition to or instead of polymerases in nanopore sequencing and can be loaded by the techniques disclosed herein. Complexes of these enzymes with nucleic acids can be loaded to nanopores as detailed herein, and the nucleic acid or enzyme component of the complex can be attached to or proximal to the nanopore. The nucleotide sequence of the nucleic acid can be determined as the nucleic acid traverses the nanopore. Methods of single molecule nanopore sequencing are known in the art and disclosed for example in US Patent Publ. Nos. 2013/0327644 and 2014/0051068, which are hereby incorporated by reference for all purposes and in particular for all teachings, written description, figures, and figure legends related to nanopore sequencing.

The methods disclosed herein can further include computer implemented processes, and/or software incorporated onto a computer readable medium instructing such processes, as set forth in greater detail below. As such, signal data generated by the reactions and optical systems disclosed herein is input or otherwise received into a computer or other data processor, and subjected to one or more of the various process steps or components set forth below. Once these processes are carried out, the resulting output of the computer implemented processes may be produced in a tangible or observable format, e.g., printed in a user readable report, displayed upon a computer display, or it may be stored in one or more databases for later evaluation, processing, reporting or the like, or it may be retained by the computer or transmitted to a different computer for use in configuring subsequent reactions or data processes.

Computers for use in carrying out the processes of this disclosure can range from personal computers such as PC or Macintosh® type computers, to workstations, laboratory equipment, or high speed servers or Cloud, running UNIX, LINUX, Windows®, or other systems. Logic processing of the disclosed methods may be performed entirely by general purposes logic processors (such as CPU's) executing software and/or firmware logic instructions; or entirely by special purposes logic processing circuits (such as ASICs) incorporated into laboratory or diagnostic systems or camera systems which may also include software or firmware elements; or by a combination of general purpose and special purpose logic circuits. Data formats for the signal data may comprise any convenient format, including digital image based data formats, such as JPEG, GIF, BMP, TIFF, or other convenient formats, while video based formats, such as avi, mpeg, mov, rmv, or other video formats may be employed. The software processes of the disclosed methods may generally be programmed in a variety of programming languages including, e.g., Matlab, C, C++, C#, NET, Visual Basic, Python, JAVA, CGI, and the like.

In some cases, the compositions, methods, and systems disclosed herein can be used as part of an integrated sequencing system, for example, as disclosed in US 20120014837—Illumination of Integrated Analytical Systems, US 20120021525—Optics Collection and Detection System and Method, US 20120019828—Integrated Analytical System and Method, 61/660,776 filed Jun. 17, 2012—Arrays of Integrated Analytical Devices and Methods for Production, and US 20120085894—Substrates and Optical Systems and Methods of Use Thereof which are incorporated herein by reference in their entirety for all purposes. Suitable sequencing systems are commercially available, e.g., from Pacific Biosciences of California.

In certain embodiments, the sequencing compositions disclosed herein will be provided in whole, or in part, in kit form enabling one to carry out the processes disclosed herein. Such kits will typically comprise one or more components of the reaction complex, such as the polymerase enzyme and primer sequences. Such kits will also typically include buffers and reagents for loading the polymerase and/or a template as in the processes disclosed herein. The kits will also optionally include other components for carrying out sequencing applications in accordance with those methods disclosed herein. In particular, such kits may include ZMW array substrates for use in observing individual reaction complexes as disclosed herein.

In addition to the various components set forth herein, the kits will typically include instructions for combining the various components in the amounts and/or ratios set forth herein, to carry out the desired processes, as also disclosed or referenced herein, e.g., for loading polymerase enzyme complexes, immobilizing polymerase enzyme complexes, and/or performing sequence by incorporation reactions.

### EXAMPLES

It is understood that the examples and embodiments disclosed herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1: Evaluation of Nonionic Surfactants for Direct Loading of Amplicon DNA into ZMW Arrays without Surface Prewetting

To evaluate whether nonionic surfactants can enable direct sample loading into ZMW arrays without the conventional prewetting step, a comparative experiment was performed using a mixed amplicon library and various surfactant formulations. The goal was to assess whether new surfactant additives, such as Tergitol 15-S-9, could enable efficient complex immobilization and high sequencing performance while reducing reagent consumption and procedural complexity.

### Materials

The DNA template consisted of a mixture of amplicon-based SMRTbell® libraries (electronic templates; ET) containing fixed insert lengths of 12 kb, 15 kb, and 20 kb, with an average insert size of approximately 16 kb. All samples were formulated as ready-to-sequence libraries.

Surfactants tested included Tergitol 15-5-9 (100%, Millipore Sigma), Silwet L-77 (100%, Fisher Scientific), and Tween-20 (10%, VWR International). Final surfactant concentrations in the mixed sample were as follows:
Control: Tween-20 only at 0.035% v/v
Sample with 0.25% Silwet L-77 spike-in
Sample with 0.25% Tergitol 15-S-9 spike-in
Prewetted standard: 0.25% Silwet L-77 in wetting buffer; sample contained 0.035% Tween-20

All samples were mixed in identical buffer compositions comprising Tris, potassium acetate (KOAc), strontium acetate (SrOAc), PEG 8000, Tween-20, dNTPs or fluorescent analogs, an oxygen scavenging system (PCA/PCD), and a triplet state quencher.

The flow cells used were standard PacBio® Revio SMRT Cells. Sequencing was performed using a Revio instrument and Reagent Plate PN 102-118-800 (Revio v1 chemistry).

### Methods

For all conditions, 80 μL of 100 μM DNA sample was combined with additional reagent components using PacBio's automated SMRTCell prep protocol, yielding a final sample volume of 220 μL and a DNA concentration of 36 μM. For the direct sample delivery conditions, 95 μL of this sample was loaded directly into a dry flow cell. For the prewetted condition, 190 μL was used to fully exchange the wetting buffer and achieve uniform sample distribution.

In the prewetted condition, the dry flow cell was first treated with 160 μL of wetting buffer containing 0.25% Silwet L-77, followed by two washes with an identical buffer lacking Silwet. In the direct loading conditions, these prewetting and wash steps were omitted entirely.

All loading steps were carried out at ambient temperature (−20° C.), with reagents maintained at 4° C. prior to use. The Revio platform's adaptive loading system was employed to monitor real-time loading progression. Immobilization of complexes occurred over a 65-minute interval.

After loading, a 24-hour sequencing movie was acquired. Base calling and ZMW classification were performed using standard Revio pipeline tools. ZMWs were classified as Empty (P0), Productive (P1), or Undetermined (P2) based on polymerase activity. A ZMW was classified as P1 (loaded and productive) if it met the following criteria: at least 50 bp of read length, a base rate between 0.5 and 3.5 bp/s, and a G-channel signal-to-noise ratio (SNR) greater than 2.5. HiFi reads were defined as consensus sequences generated from three or more full passes through the insert. For comparisons between conditions, P1% was used to represent the percentage of ZMWs classified as productive and contributing to sequencing yield.

### Results

The comparative results are summarized in Table 1. Sample spiked with Tergitol 15-S-9 achieved 46 ZMW loading using only 100 μL of input volume—matching the 450 loading obtained with the 200 μL prewetted standard. Silwet spike-in (0.250) yielded only 5% loading. The control condition (Tween-20 only, 0.0350%) yielded 1000 loading.

TABLE 1
Effect of detergent on sample direct wetting sequencing performance

| | | | | | Read | HiFi |
|---|---|---|---|---|---|---|
| | Loading | | Loading | Base | Length | Yield |
| Conditions | Volume | Methods | (P1%) | Rate | (kbp) | (gb) |
| Sample with | 100 μl | Direct loading | 5% | 2.7 | 105.8 | 11.5 |
| 0.25% SilWet | | without prewetting | | | | |
| L-77 Spike-in | | the surface | | | | |
| Sample with | 100 μl | Direct loading | 46% | 2.5 | 87.0 | 87.3 |
| 0.25% Tergitol | | without prewetting | | | | |
| Spike-in | | the surface | | | | |
| Sample | 100 μl | Direct loading | 10% | 2.4 | 96.4 | 20.3 |
| without | | without prewetting | | | | |
| surfactant | | the surface | | | | |
| Sample | 200 μl | Loading on | 45% | 2.6 | 86.8 | 83.2 |
| without | | surface prewetted | | | | |
| surfactant | | with SilWet L-77 | | | | |

These results represent single runs per condition. The Silwet prewetted, Silwet spike-in, and no-surfactant direct load conditions replicate prior internal findings. The test in this study was the use of 0.250% Tergitol 15-S-9 in direct loading conditions, evaluated head-to-head with historical benchmarks.

### Conclusions

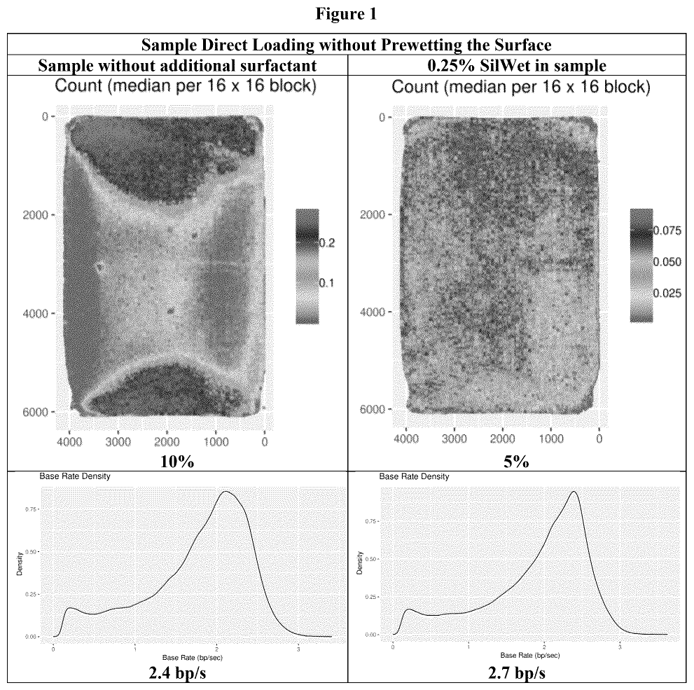

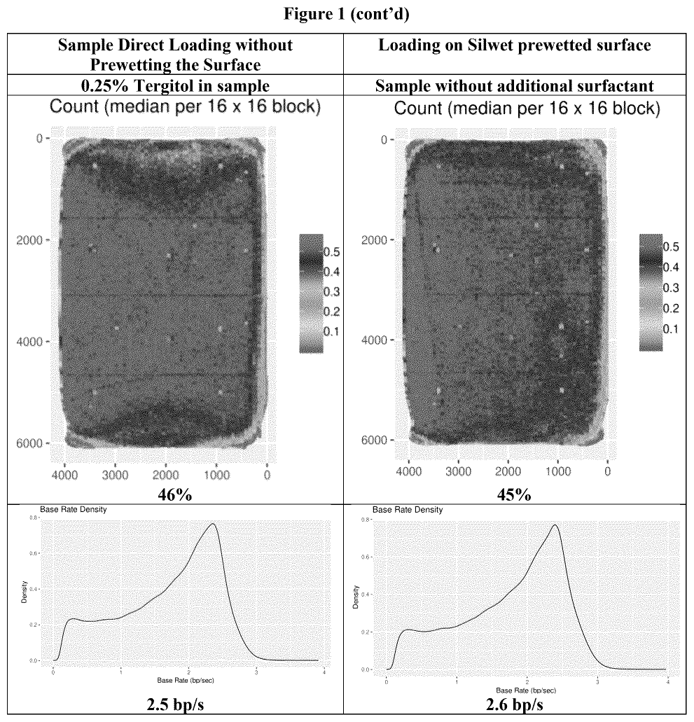

This example demonstrates that Tergitol 15-S-9, when included in the loading buffer at 0.250% (v/v), supports high-efficiency complex immobilization in ZMW arrays without the need for surface prewetting. See FIG. 1 and Table 1. The loading efficiency and HiFi yield were comparable to those achieved using the conventional prewetting protocol, but required only half the sample volume (95 μL vs. 190 μL).

In contrast, spiking Silwet L-77 directly into the sample significantly impaired loading efficiency. The standard sample formulation without prewetting (Tween-20 only) also produced suboptimal loading and altered sequencing photonic properties.

The elimination of the prewetting step simplifies sample preparation, reduces reagent consumption, and avoids laminar dilution effects observed in flow cells. This results in more uniform PEG concentration across the array and supports preferential loading of longer DNA complexes, which in turn contributes positively to overall HiFi yield.

### Example 2: Optimization of Tergitol 15-S-9 Concentration for Enhanced Loading and Sequencing Performance without Prewetting

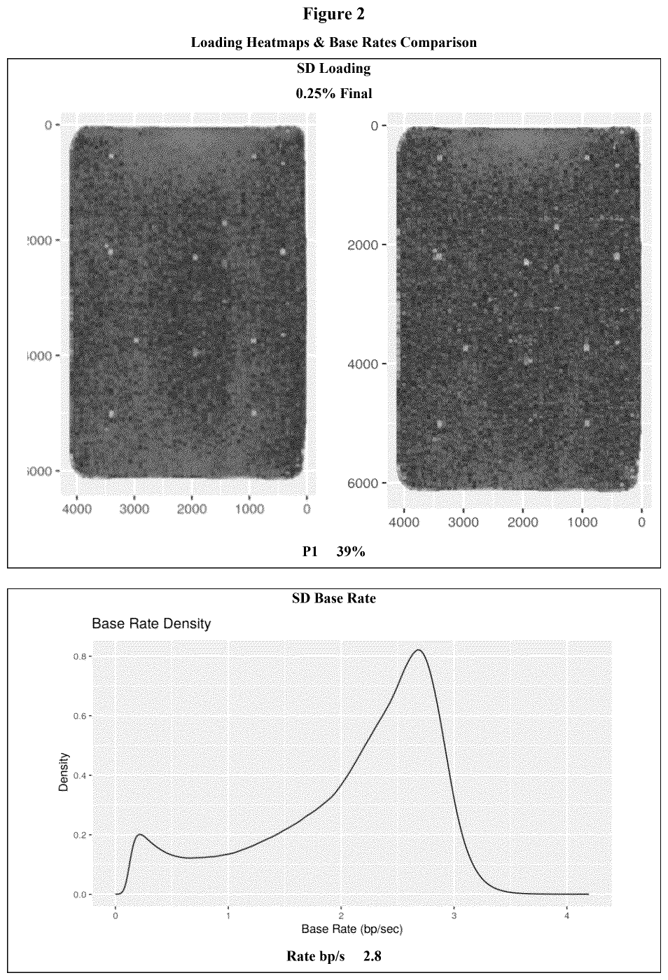

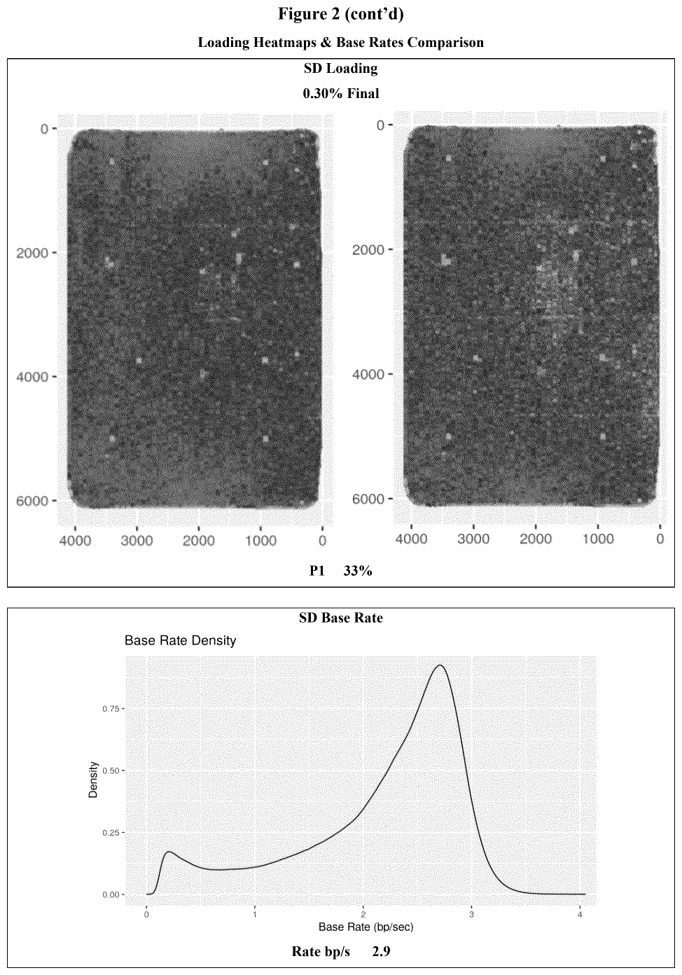

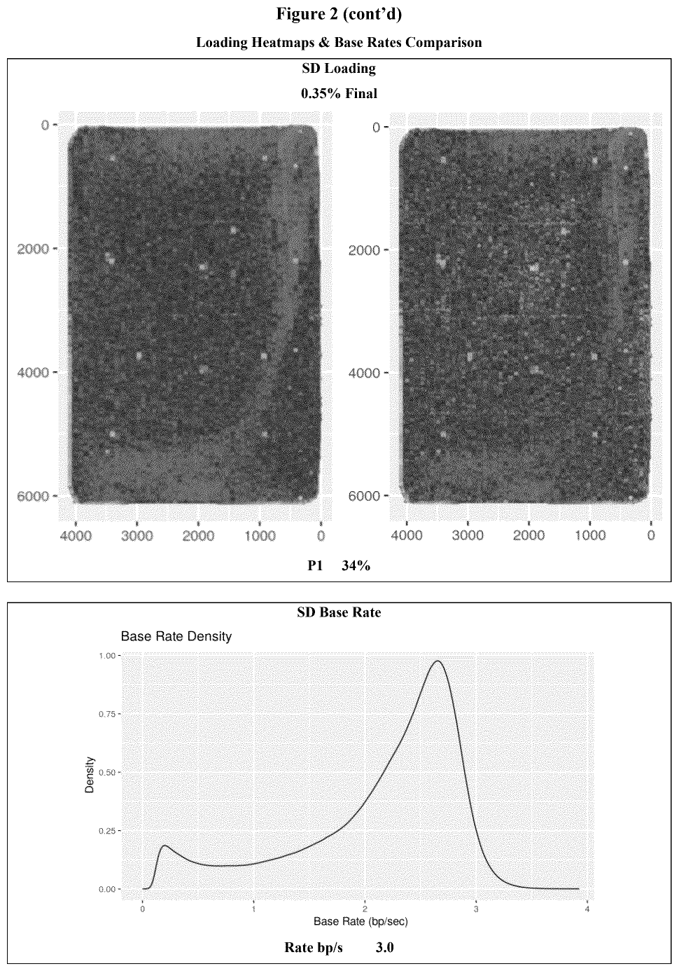

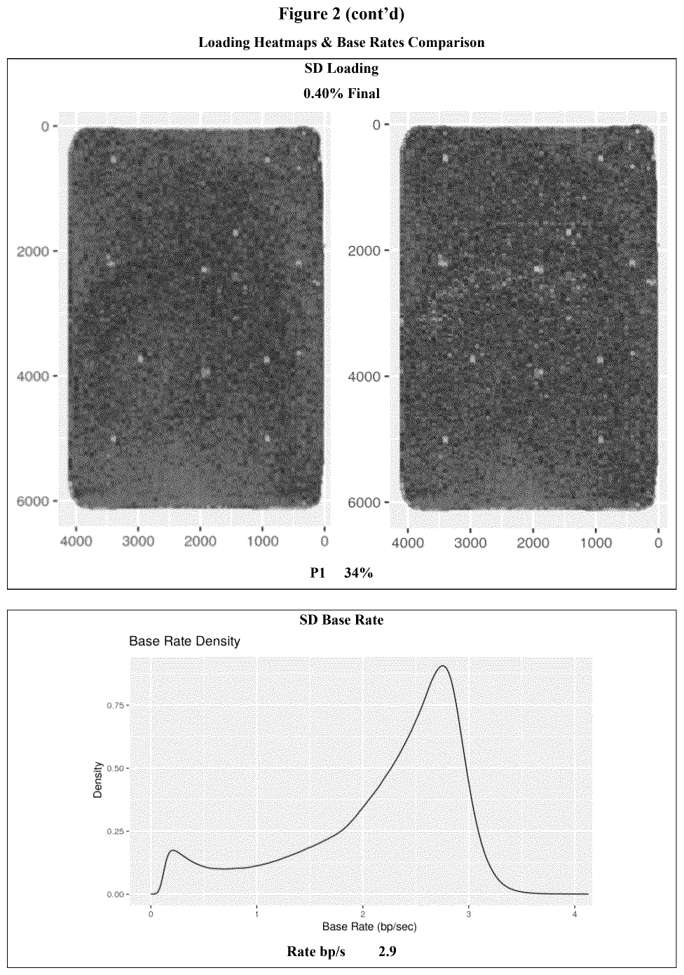

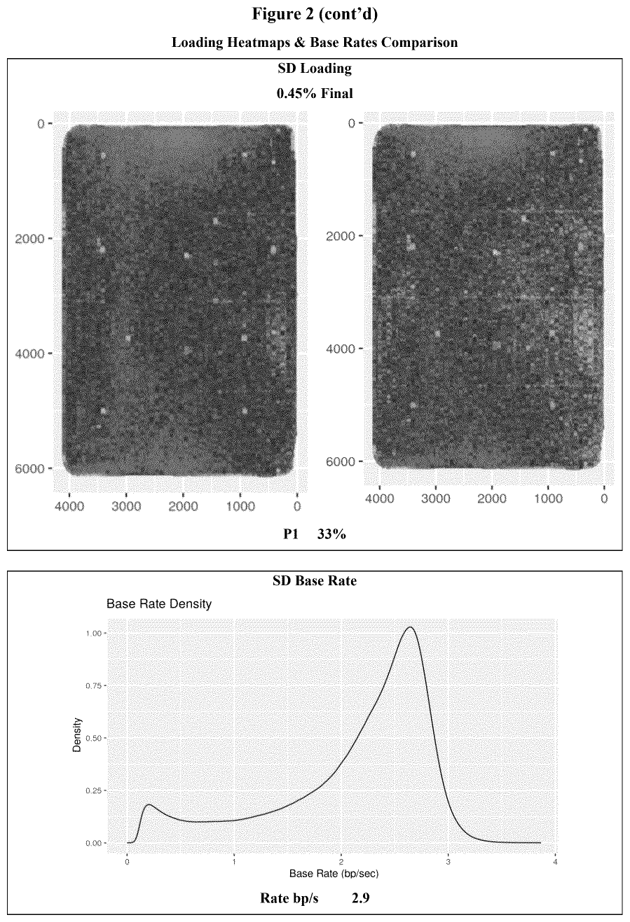

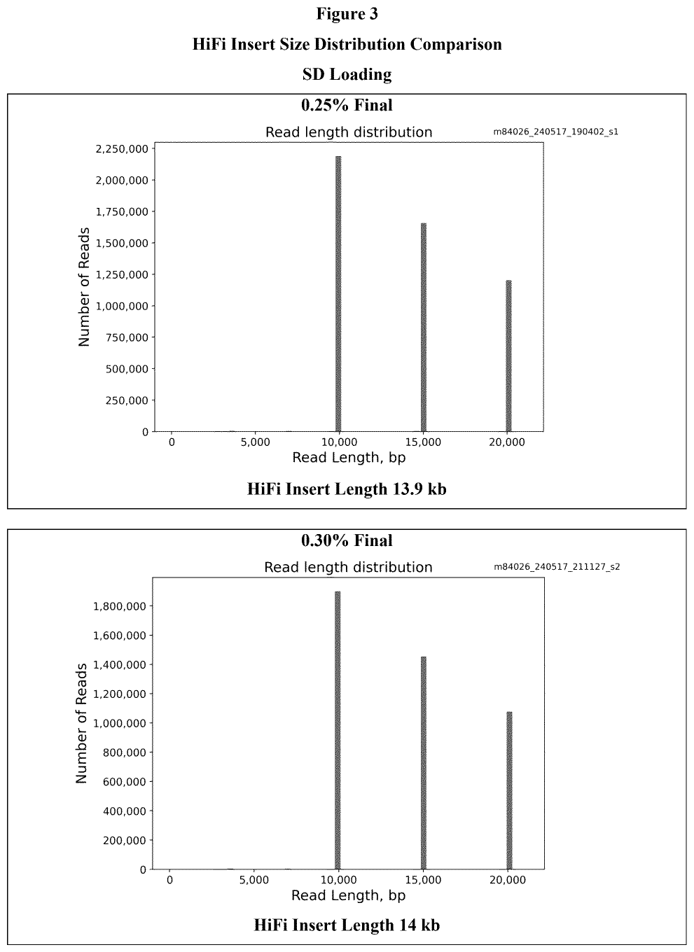

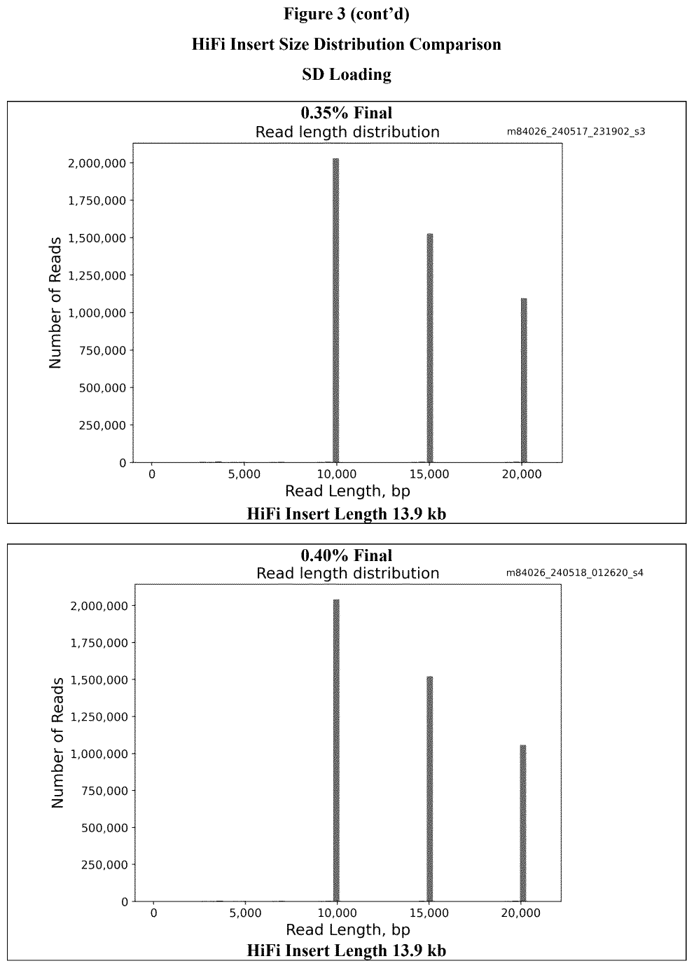

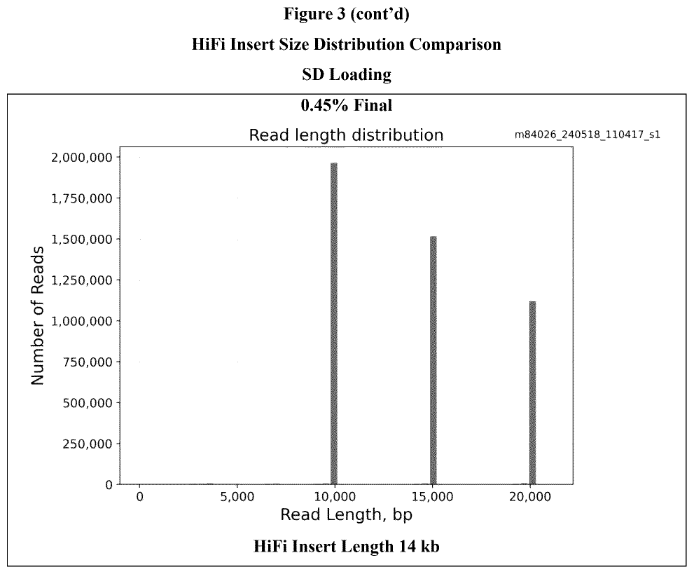

A titration study was conducted to evaluate the effect of varying concentrations of Tergitol 15-S-9 (0.25%, 0.30%, 0.35%, 0.40%, and 0.45% v/v) in a PEG 8000-containing loading buffer on direct sample loading into ZMW arrays. As shown in FIG. 2 and FIG. 3, these concentrations were selected to identify an optimal range that balances complex immobilization efficiency, sequencing kinetics, and overall yield, without requiring surface prewetting.

### Materials and Methods

The DNA sample, surfactant sources, buffers, flow cells, instrument (Revio), and automated preparation workflow were identical to those disclosed in Example 1.

In this experiment, Tergitol 15-S-9 was spiked into the PEG-containing reagent component at concentrations adjusted to yield final concentrations of 0.25%, 0.30%, 0.35%, 0.40%, and 0.45% (v/v) in the mixed sample. Each sample was mixed to a final DNA concentration of 36 μM in 220 μL, and 95 μL was delivered directly to a dry Revio SMRT Cell. No prewetting or washing steps were performed before sample delivery.

All other loading, incubation, and sequencing parameters were as disclosed in Example 1, except that the sequencing movies were collected for 12 hours instead of 24 hours, using Revio v1 chemistry (PN 102-118-800).

### Results

The results of the titration are summarized in Table 2. Across the tested concentration range, Tergitol 15-S-9 supported stable loading and consistent base rate performance. The highest base rate (3.0 bp/s) was observed at 0.35%, with corresponding loading of 34%. The highest overall loading efficiency (39%) occurred at 0.25%, although this condition did not produce the longest polymerase read length. This outcome aligns with the general inverse relationship between loading and read length, where higher occupancy can result in shorter average reads. Overall, concentrations between 0.25% and 0.40% (v/v) maintained strong sequencing performance across all evaluated metrics.

TABLE 2
Effect of Tergitol 15-S-9 concentration on sequencing performance

| | | | Polymerase |
|---|---|---|---|
| Tergitol 15-S-9 | Loading | Base Rate | Read Length |
| (v/v) | (P1%) | (bp/s) | (mean, bp) |
| 0.25% | 39% | 2.8 | 61,300 |
| 0.30% | | 2.9 | 63,300 |
| 0.35% | 34% | 3.0 | 65,000 |
| 0.40% | 34% | 2.9 | 66,000 |
| 0.45% | 33% | 2.9 | 65,100 |

Loading efficiencies were in a narrow range (33-39%), with the highest observed at 0.25%. All runs used equivalent DNA input and conditions, with only the Tergitol concentration varied. No loading artifacts or changes in sequencing quality were observed.

### Conclusions

Tergitol 15-5-9 concentrations between 0.30% and 0.40% (v/v) provided the most favorable balance of performance, achieving the highest polymerase read lengths and base incorporation rates without compromising photonic or enzymatic activity. The peak polymerase read length (˜66,000 bp) was observed at 0.40%, while the highest base rate (3.0 bp/s) occurred at 0.35%. These differences are within the expected range of experimental variability.

Sequencing performance was broadly comparable across the full concentration range of 0.25%-0.45%. However, the performance gains appeared to plateau above 0.40%, suggesting a concentration-dependent upper limit to performance, possibly due to micelle formation or saturation of the array surface by the surfactant. Concentrations below 0.25% were not tested in this study, as previous experiments had indicated reduced loading efficiency at lower levels.

Overall, this titration study demonstrated that a surfactant, such as Tergitol 15-5-9, is highly compatible with direct sample loading workflows, supporting efficient complex immobilization while enabling the elimination of surface prewetting steps and reduction of sample input volume, all without compromising sequencing quality metrics.

### Example 3: Comparative Evaluation of Tergitol 15-S-9 and EcoSurf EH9 for Direct Sample Loading without Prewetting

This example evaluates the performance of two nonionic surfactants, Tergitol 15-S-9 and EcoSurf EH9, across a range of concentrations when used for direct sample loading into ZMW arrays without prewetting. The study compared loading efficiency, sequencing kinetics, and polymerase read length to identify robust and concentration-tolerant surfactants suitable for broad application across different library types.

### Materials and Methods

The DNA library, buffer composition, reagent components, flow cells (Revio SMRT Cells), sequencing chemistry (Revio v1, PN 102-118-800), and automated sample preparation protocol were identical to those disclosed in Example 1. All sample mixes included PEG 8000 and were prepared using standard PacBio protocols.

Tergitol 15-S-9 (“Tergitol”) and EcoSurf EH-9 (“EcoSurf”) were tested by titrating detergent into a PEG-containing sample mix component. Both detergents were prepared at 100% stock and spiked into the mix at varying volumes to achieve final concentrations in the complete sample of:
Tergitol 15-S-9: 0.01%, 0.03%, 0.05%, 0.10%, 0.25%, 0.50% (v/v); and
EcoSurf EH-9: 0.10%, 0.25%, 0.50% (v/v).

All conditions used 95 μL of prepared sample per SMRT Cell. No surface prewetting or wash steps were used for experimental conditions. Control samples included a Silwet L-77 prewetted condition and a Silwet L-77 spike-in (0.25%) in the sample itself, which is known to impair loading. Tergitol and EcoSurf titrations were performed on the same day using aliquots from the same DNA sample batch; each titration set was run on a separate Revio instrument to accommodate the number of conditions.

Sequencing movies were collected for 24 hours under standard Revio v1 conditions. Base rate, polymerase read length (mean), and loading percentage (P1 ZMWs) were extracted from post-run data analysis. P1 ZMWs were defined using the same thresholds disclosed in Example 1.

### Results

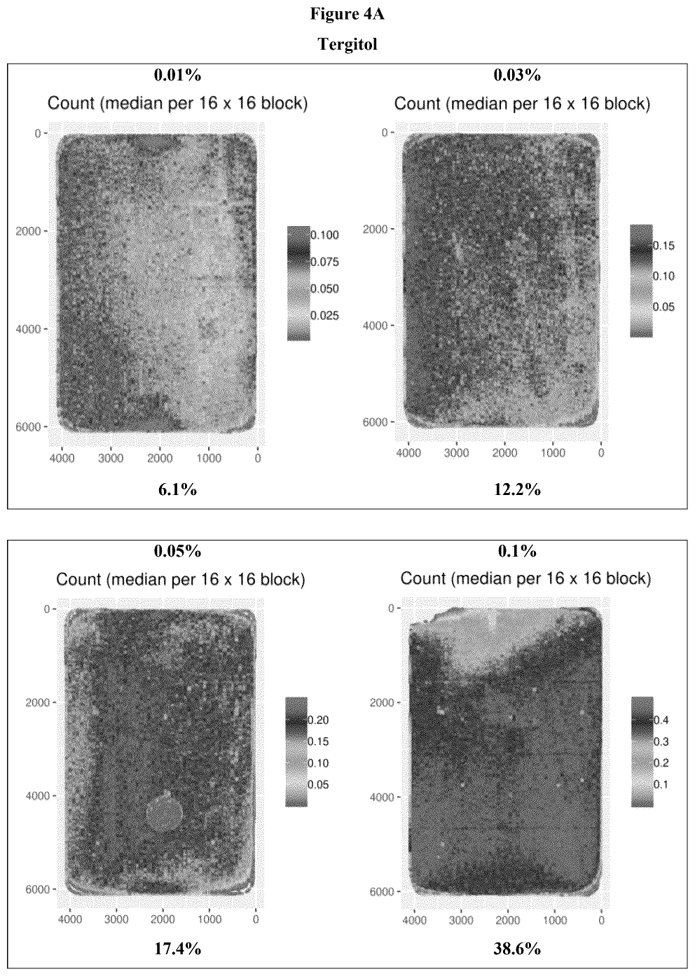

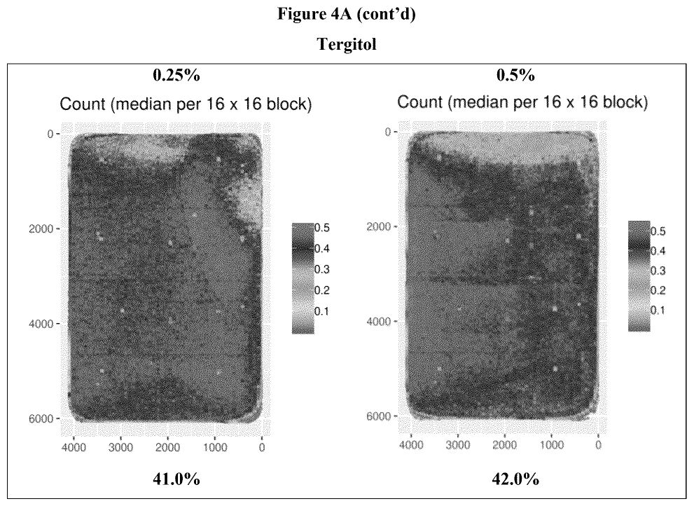

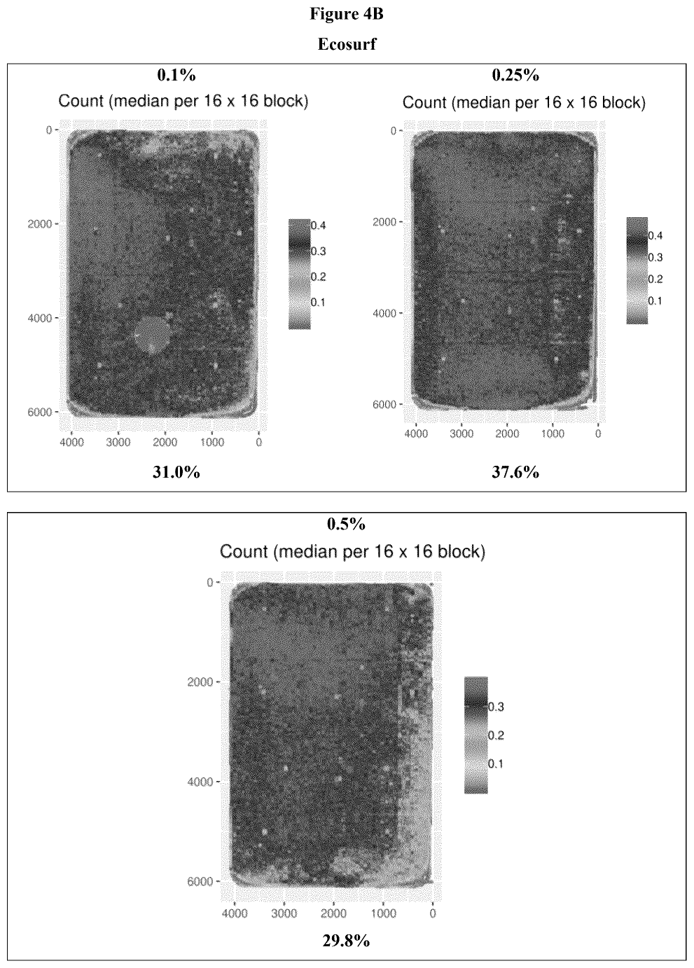

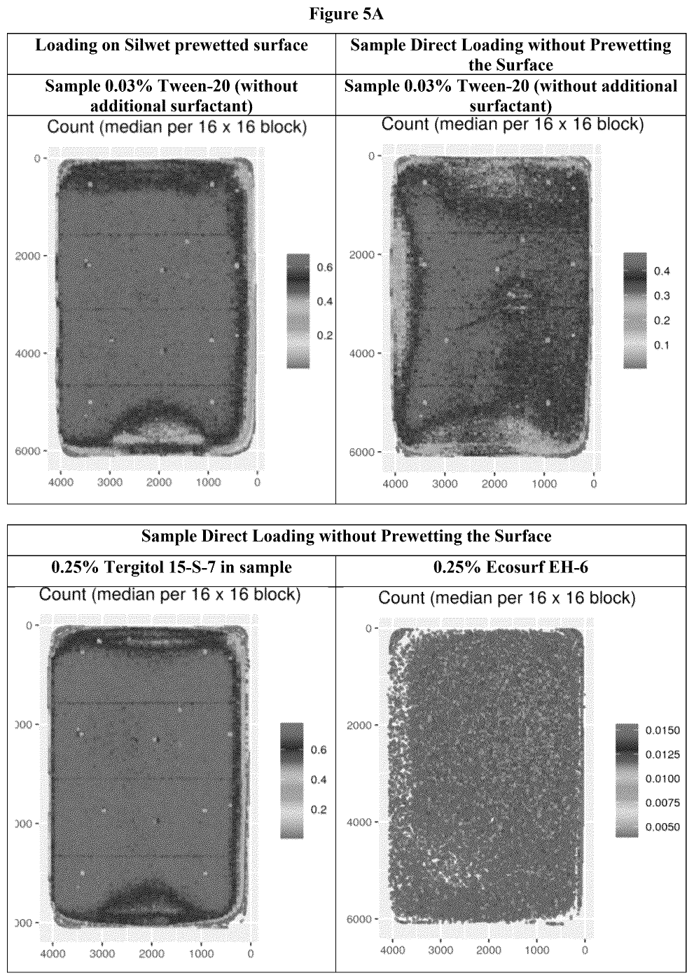

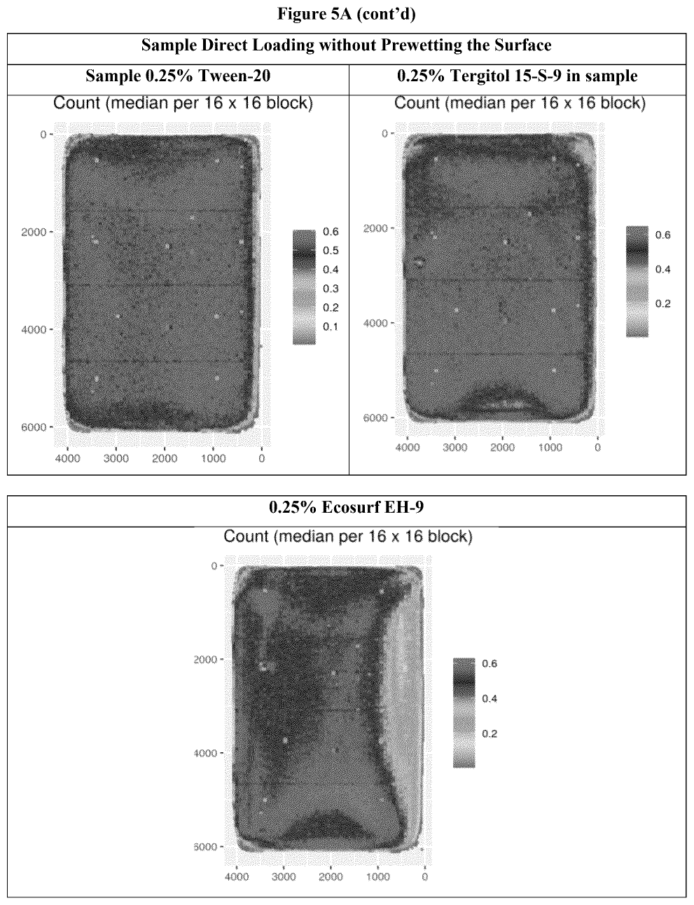

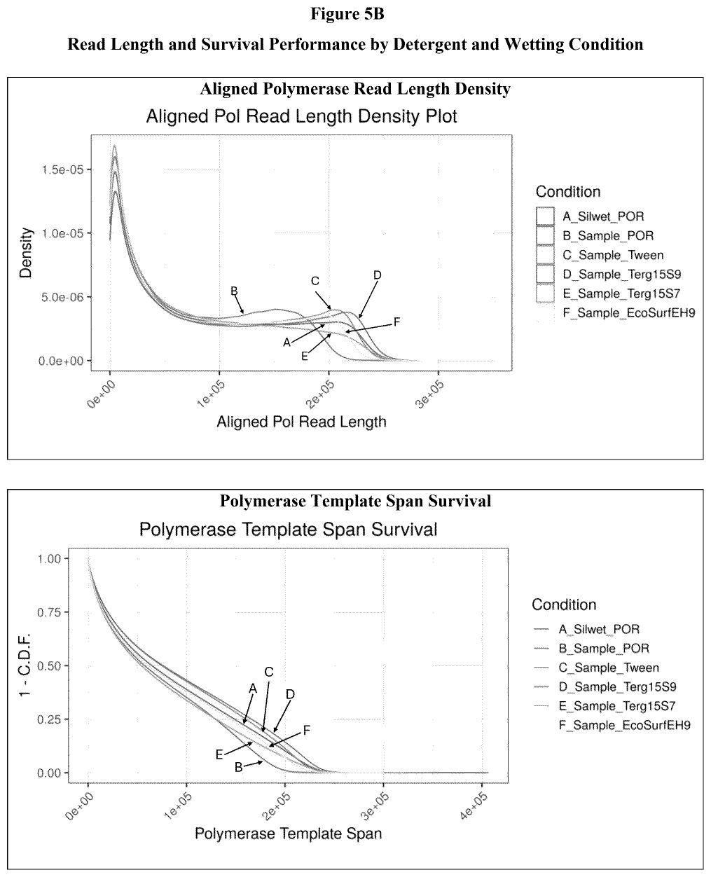

Results for the Tergitol and EcoSurf titrations are summarized in Table 3 and visualized in FIGS. 4A and 4B. These figures illustrate the comparative ZMW loading efficiencies and polymerase read lengths observed across multiple surfactant concentrations. At their optimal concentrations, both detergents matched or exceeded the performance of the Silwet prewetting controls; however, EcoSurf performance declined at non-optimal concentrations. Tergitol showed a concentration-dependent increase in loading up to 0.25%, beyond which performance plateaued at the highest tested concentration of 0.50%. EcoSurf showed the highest performance at 0.25% but declined at both lower and higher concentrations.

TABLE 3
Comparison of Tergitol and EcoSurf across multiple
concentrations for direct sample loading

| | | | | Polymerase |
|---|---|---|---|---|
| | Concentration | Loading | Base Rate | Read Length |
| Surfactant | (v/v) | (% P1) | (bp/s) | (mean, bp) |
| Silwet (prewet - | 0.25% | 28.2 | 2.7 | 99,924 |
| Tergitol control) | | | | |
| Silwet (0.25% | 0.25% | 14.9 | 2.5 | 109,925 |
| Spike-in) | | | | |
| Tergitol | 0.01% | 6.1 | 2.5 | 94,652 |
| Tergitol | 0.03% | 12.2 | 2.7 | 102,716 |
| Tergitol | 0.05% | 17.4 | 2.9 | 105,377 |
| Tergitol | 0.10% | 38.6 | 2.9 | 104,413 |
| Tergitol | 0.25% | 41.0 | 2.7 | 97,000 |
| Tergitol | 0.50% | 42.0 | 2.6 | 98,880 |
| Silwet (prewet - | 0.25% | 37.4 | 2.8 | 95,657 |
| EcoSurf control) | | | | |
| EcoSurf | 0.10% | 31.0 | 2.6 | 83,032 |
| EcoSurf | 0.25% | 37.6 | 2.7 | 98,332 |
| EcoSurf | 0.50% | 29.8 | 2.6 | 93,936 |

Note:
Silwet prewetting controls were run separately for the Tergitol and EcoSurf titrations, each on a different Revio instrument

### Conclusions

Tergitol enabled robust direct sample loading across a broad concentration range, achieving >40% ZMW loading at concentrations >0.25% and maintaining stable sequencing performance even at 0.50%. No performance degradation was observed at higher concentrations, making Tergitol well-suited for a variety of library fragment sizes, including high-PEG formulations used for short inserts where the final detergent concentration may be higher due to formulation requirements.

In addition, EcoSurf achieved its best results at 0.25%, although decreased loading and shorter read lengths were observed at both 0.10% and 0.50%. This trend indicates a narrower effective range, with performance declining at higher concentrations potentially arising from interference with polymerase activity, DNA condensation, or surface binding efficiency. Overall, Tergitol demonstrated greater compatibility and reliability as a surfactant for direct loading workflows, supporting consistent complex immobilization, long read lengths, and high sequencing productivity across a wide range of conditions.

### Example 4: Detergent Screening Using a 16 kb HG002 DNA Library for Direct Sample Loading without Prewetting

This example demonstrates a detergent screening experiment to evaluate the impact of various surfactants on direct sample loading and sequencing performance using a 16 kb mean fragment size HG002 human DNA library. The study compared ZMW loading efficiency, polymerase read length, and survival across multiple detergent conditions to identify formulations that optimize both loading and sequencing outcomes without requiring surface prewetting.

### Materials and Methods

All buffer compositions, reagents, and flow cell conditions were identical to those disclosed in Example 1, including the use of PEG 8000 and Revio SMRT Cells with Revio v1 sequencing chemistry (PN 102-118-800). The library was prepared from human genomic DNA (HG002) sheared to an average of 16 kb and converted to SMRTbell-libraries (DNA w/adapters, primers, polymerase) using standard protocols.

All detergent-containing conditions were prepared by spiking concentrated detergents into a PEG-containing sample mix component. Unless otherwise noted, detergents were added as 100% stock solutions to achieve a final sample concentration of 0.25% (v/v). EcoSurf EH6 was an exception and was used as a 50% stock. The following conditions were tested:
Silwet L-77 prewetting control (standard PacBio SMRTcell prep)
0.03% Tween-20 (standard sample used as a baseline condition with no prewetting)
0.25% Tween-20
0.25% Tergitol 15-S-7
0.25% Tergitol 15-S-9
0.25% EcoSurf EH6
0.25% EcoSurf EH9

Direct sample loading conditions used 95 μL of prepared sample. The prewetted control used 190 μL to displace the wetting buffer. Each condition was loaded onto a Revio SMRT Cell and sequenced for 24 hours. All samples were derived from the same master dilution and loaded on the same day to minimize variability.

### Results

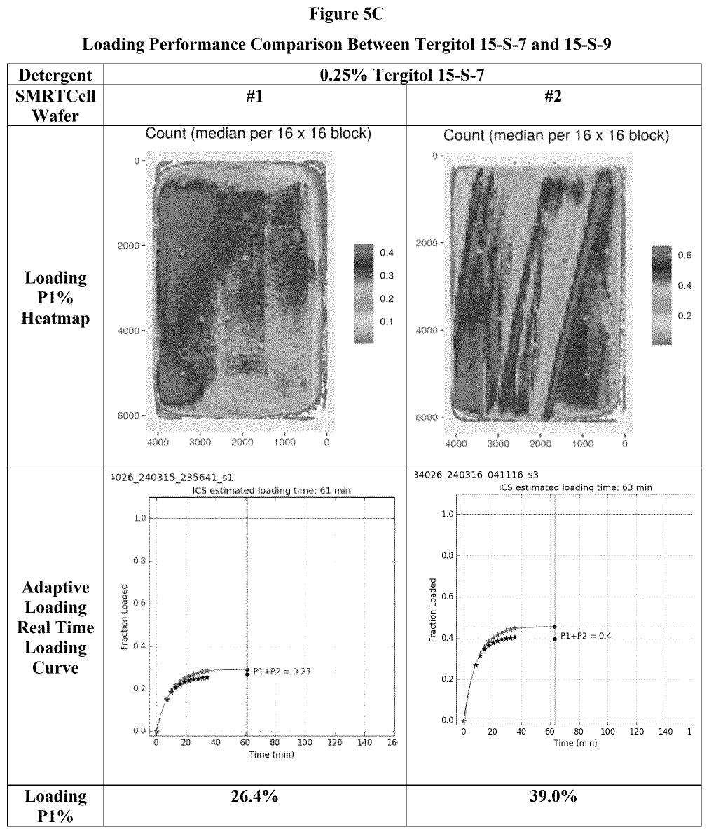

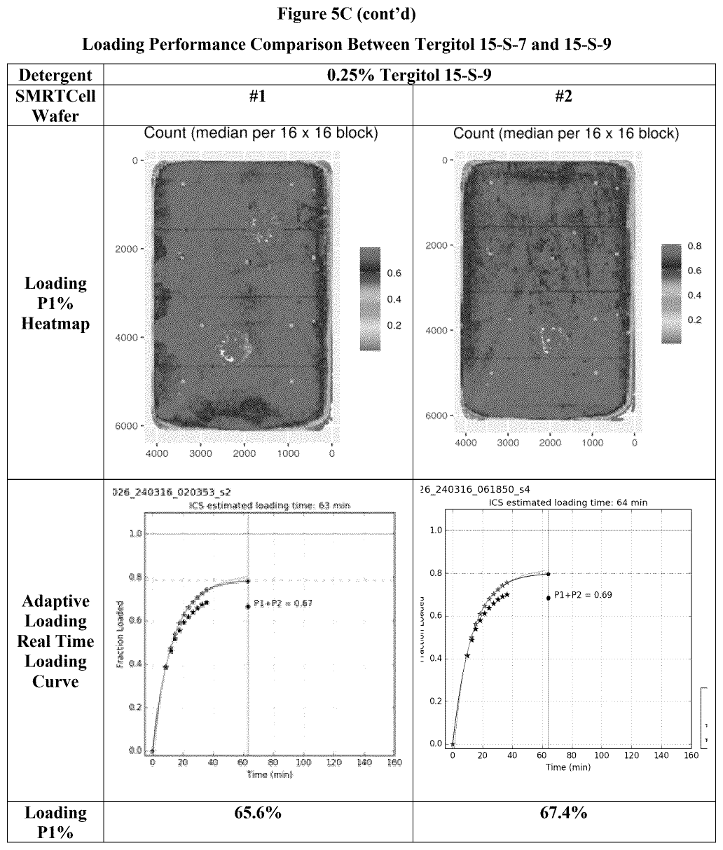

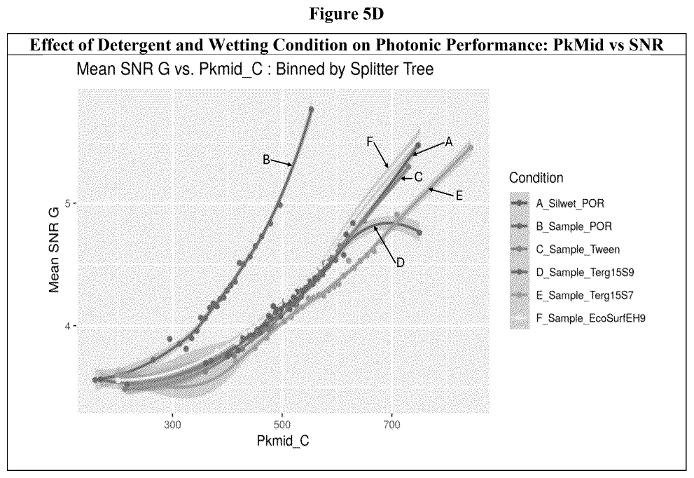

Loading performance (P1 ZMW occupancy) and polymerase read length metrics are summarized in Table 4 and visualized in FIGS. 5A through 5D.

Among the detergents tested, 0.25% Tergitol 15-S-9 and 0.25% Tween-20 achieved P1 loading comparable to or exceeding the Silwet prewetting control, while also improving read length and polymerase survival. Notably, these performance gains were achieved using only 95 μL of sample per SMRT Cell, compared to the 190 μL required in the prewetted workflow, highlighting the efficiency of direct-loading formulations. Tergitol 15-S-7 showed the highest loading (63%) but was later found to exhibit variability in replicate experiments (see FIG. 5C). Tergitol 15-S-9 consistently supported high loading (54%) and long read lengths, indicating robust performance.

TABLE 4
Comparison of Detergents for Direct Sample
Loading of 16 kb HG002 DNA Library

| | Sample | Final | | Polymerase |
|---|---|---|---|---|
| | Volume | Detergent | Loading | Read Length |
| Condition | (μL) | (%) | (% P1) | (bp) |
| Silwet Prewet | 190 | 0.25% | 55% | 84,100 |
| (control) | | | | |
| 0.03% Tween-20 | 95 | 0.03% | 35% | 74,100 |
| (baseline) | | | | |
| 0.25% Tween-20 | 95 | 0.25% | 50% | 89,700 |
| Tergitol 15-S-9 | 95 | 0.25% | 54% | 92,400 |
| Tergitol 15-S-7 | 95 | 0.25% | 63% | 75,800 |
| EcoSurf EH9 | 95 | 0.25% | 47% | 80,700 |
| EcoSurf EH6 | 95 | 0.25% (from 50% | 0% | 94,400 |
| | | stock) | | |

As shown in Table 4 and FIG. 5B, 0.25% o Tergitol 15-S-9 and 0.25% o Tween-20 achieved both high P1 loading and long polymerase read lengths. These two conditions showed a broader read length distribution, with a greater proportion of reads above 200 kb compared to other detergents. EcoSurf EH9 restored loading to near-control levels but underperformed a little in downstream metrics. EcoSurf EH6 yielded no loading, and thus no sequencing output.

### Conclusions

This detergent screen demonstrated that Tergitol 15-S-9 provides the best combination of high ZMW loading, long polymerase read lengths, and polymerase survival. All high-detergent conditions (including 0.25% o Tween-20, Tergitol variants, and EcoSurf EH9) successfully restored photonic performance, as shown by PkiMid vs. SNR trends in Figure SD. This demonstrates effective surface wetting under direct loading conditions.

Additionally, Tergitol 15-S-9 achieved consistently high ZMW loading in combination with long polymerase read lengths and robust polymerase survival, thereby outperforming other detergents across key sequencing performance metrics. Tergitol 15-S-7 also exhibited the highest loading in the experiment, but the results may be variable, with performance ranging from 260% to 67% o P1 (Figure SC).

While the foregoing disclosure has provided some details for purposes of clarity and understanding, it will be clear to one skilled in the art from a reading of this disclosure that various changes in form and detail can be made without departing from the true scope of the invention. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. A method of loading polymerase enzyme complexes into a plurality of nanoscale wells, the method comprising contacting a loading solution to a surface of a substrate comprising a plurality of nanoscale wells, wherein the loading solution comprises:
(a) one or more polymerase enzyme complexes comprising a template nucleic acid and a polymerase enzyme;
(b) one or more nonionic surfactants; and
(c) one or more crowding agents.

2. The method of claim 1, wherein the surface is hydrophobic before contacting with the loading solution.

3. The method of claim 1, wherein the surface is not prewetted before contacting with the loading solution.

4. The method of claim 1, wherein the one or more polymerase enzyme complexes are suspended in a sample mix solution.

5. The method of claim 4, wherein the sample mix solution comprises one or more surfactants.

6. The method of claim 4, wherein the sample mix solution comprises one or more nonionic surfactants.

7. The method of claim 6, wherein the nonionic surfactant is a detergent.

8. The method of claim 6, wherein the nonionic surfactant is selected from the group consisting of Triton X-100, EcoSurf, Tergitol, poloxamer, ECO Brij, Brij, n-Dodecyl β-D-maltoside, N,N-dimethyldodecylamine N-oxide, and Tween-20.

9. The method of claim 4, wherein the sample mix solution comprises one or more viscosity adjusting agents.

10. The method of claim 9, wherein the viscosity adjusting agent is selected from the group consisting of glycerol, a low molecular weight PEG, a polysaccharide such as cellulose, agar, dextrin, or trehalose, and polyvinylpyrrolidone (PVP).

11. The method of claim 4, wherein the sample mix solution comprises one or more monovalent cations, one or more divalent cations, or a combination thereof.

12. The method of claim 1, wherein the nonionic surfactant is selected from the group consisting of Triton X-100, EcoSurf, Tergitol, poloxamer, ECO Brij, Brij, n-Dodecyl β-D-maltoside, N,N-dimethyldodecylamine N-oxide, and Tween-20.

13. The method of claim 1, wherein the loading solution comprises about 0.01% (v/v), about 0.02% (v/v), about 0.03% (v/v), about 0.04% (v/v), about 0.05% (v/v), about 0.06% (v/v), about 0.07% (v/v), about 0.08% (v/v), about 0.09% (v/v), about 0.1% (v/v), about 0.15% (v/v), about 0.2% (v/v), about 0.25% (v/v), about 0.30% (v/v), about 0.35% (v/v), about 0.4% (v/v), about 0.45% (v/v), about 0.5% (v/v), about 0.55% (v/v), about 0.6% (v/v), about 0.65% (v/v), about 0.7% (v/v), about 0.75% (v/v), about 0.8% (v/v), about 0.85% (v/v), about 0.9% (v/v), about 0.95% (v/v), or about 1% (v/v) of the one or more nonionic surfactants.

14. The method of claim 1, wherein the one or more crowding agents comprise a high molecular weight PEG, polyvinylpyrrolidone (PVP), or a combination thereof.

15. The method of claim 1, wherein the crowding agent is a high molecular weight PEG.

16. The method of claim 15, wherein the PEG has a molecular weight between about 3,000 g/mol and about 40,000 g/mol, between about 5,000 g/mol and about 30,000 g/mol, between about 6,000 g/mol and about 20,000 g/mol, between about 7,000 g/mol and about 12,000 g/mol, or between about 8,000 g/mol and about 10,000 g/mol.

17. The method of claim 15, wherein the PEG is selected from the group consisting of PEG 3000, PEG 4000, PEG 5000, PEG 6000, PEG 7000, PEG 8000, PEG 9000, PEG 10000, and PEG 20000.

18. The method of claim 15, wherein the PEG is at a concentration of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM in the loading solution.

19. The method of claim 1, wherein the loading solution further comprises one or more viscosity adjusting agents.

20. The method of claim 19, wherein the loading solution comprises about 0.1% (v/v), about 0.5% (v/v), about 1% (v/v), about 1.5% (v/v), about 2% (v/v), about 2.5% (v/v), about 3% (v/v), about 3.5% (v/v), about 4% (v/v), about 4.5% (v/v), about 5% (v/v), about 5.5% (v/v), about 6% (v/v), about 6.5% (v/v), about 7% (v/v), about 7.5% (v/v), about 8% (v/v), about 8.5% (v/v), about 9% (v/v), about 9.5% (v/v), or about 10% (v/v) of the viscosity adjusting agent.

21. The method of claim 19, wherein the viscosity adjusting agent is selected from the group consisting of glycerol, a low molecular weight PEG, a polysaccharide such as cellulose, agar, dextrin, or trehalose, and polyvinylpyrrolidone (PVP).

22. The method of claim 1, wherein the loading solution further comprises one or more monovalent cations, one or more divalent cations, or a combination thereof.

23. The method of claim 22, wherein the monovalent cation is Na⁺, K⁺, or a combination thereof.

24. The method of claim 22, wherein the divalent cation is Sr²⁺.

25. The method of claim 22, wherein the loading solution comprises KOAc at a concentration of about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM.

26. The method of claim 22, wherein the loading solution comprises SrOAc at a concentration of about 10 μM, about 20 μM, about 30 μM, about 40 μM, about 50 μM, about 60 μM, about 70 μM, about 80 μM, about 90 μM, about 100 μM, about 110 μM, about 120 μM, about 130 μM, about 140 μM, about 150 μM, about 160 μM, about 170 μM, about 180 μM, about 190 μM, about 200 μM, about 210 μM, about 220 μM, about 230 μM, about 240 μM, about 250 μM, about 260 μM, about 270 μM, about 280 μM, about 290 μM, or about 300 μM.

27. A loading solution for loading polymerase enzyme complexes into a plurality of nanoscale wells, the loading solution comprising:
(a) one or more nonionic surfactants; and
(b) one or more crowding agents.

28. The loading solution of claim 27, further comprising one or more polymerase enzyme complexes comprising a template nucleic acid and a polymerase enzyme.

29. The loading solution of claim 27 or claim 28, wherein the surfactant is a nonionic surfactant.

30. The loading solution of claim 29, wherein the nonionic surfactant is a detergent.

31. The loading solution of claim 29, wherein the nonionic surfactant is selected from the group consisting of Triton X-100, EcoSurf, Tergitol, poloxamer, ECO Brij, Brij, n-Dodecyl 3-D-maltoside, N,N-dimethyldodecylamine N-oxide, and Tween-20.

32. The loading solution of claim 27 or 28, wherein the crowding agent is a high molecular weight PEG.

33. The loading solution of claim 32, wherein the PEG has a molecular weight between about 3,000 g/mol and about 40,000 g/mol, between about 5,000 g/mol and about 30,000 g/mol, between about 6,000 g/mol and about 20,000 g/mol, between about 7,000 g/mol and about 12,000 g/mol, or between about 8,000 g/mol and about 10,000 g/mol.

34. The loading solution of claim 32, wherein the PEG is selected from the group consisting of PEG 3000, PEG 4000, PEG 5000, PEG 6000, PEG 7000, PEG 8000, PEG 9000, PEG 10000, and PEG 20000.

35. The loading solution of claim 27 or claim 28, further comprising: one or more viscosity adjusting agents, one or more monovalent cations, one or more divalent cations, or a combination thereof.

36. The loading solution of claim 35, wherein the viscosity adjusting agent is selected from the group consisting of glycerol, a low molecular weight PEG, a polysaccharide such as cellulose, agar, dextrin, or trehalose, and polyvinylpyrrolidone (PVP).
